(19) United States
(12) Patent Application Publication
(10) Pub. No.: US 2003/0152945 A1
(43) Pub. Date: Aug. 14, 2003

(54) Cell cycle progression proteins

(75) Inventors: Peter Deak, Cambridge (GB); David Moore Glover, Sandy (GB); Carol Midgley, Milton Keynes (GB)

Correspondence Address: LAHIVE & COCKFIELD, 28 STATE STREET, BOSTON, MA, 02109, US

(73) Assignee: Cyclacel Limited, Dundee (GB)

(21) Appl. No.: 10/161,051

(22) Filed: May 30, 2002

Related U.S. Application Data
(63) Continuation-in-part of application No. PCT/GB01/01297, filed on Mar. 23, 2001.

(30) Foreign Application Priority Data
Mar. 24, 2000 (GB) 0007268.6

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 536/23.2

(57) ABSTRACT

Polynucleotides encoding a number of Drosophila gene products are provided. Polynucleotide probes derived from these nucleotide sequences, polypeptides encoded by the polynucleotides and antibodies that bind to the polypeptides are also provided.

### RELATED APPLICATION

This application is a continuation-in-part application PCT/GB01/01297 filed on Mar. 23, 2001, pending, published pursuant to PCT Article 21, in English, and application GB 0007268.6 filed Mar. 24, 2000, the entire contents of which are incorporated herein by this reference.

### BACKGROUND OF THE INVENTION

The present invention relates to a number of genes implicated in the processes of cell cycle progression, including mitosis and meiosis.

We have now identified a large number of genes in Drosophila, mutations in which disrupt cell cycle progression, for example the processes of mitosis and/or meiosis. We have determined the phenotypes of these mutations and recovered nucleotide sequences associated with the corresponding genes. Many of these nucleotide sequences correspond to protein open reading frames (ORFs) present in the Drosophila genome.

Accordingly the present invention provides in one aspect a polynucleotide selected from: (a) polynucleotides comprising any one of the nucleotide sequences set out in Examples 1 to 70 or the complement thereof; (b) polynucleotides comprising a nucleotide sequence capable of hybridising to the nucleotide sequences set out in Examples 1 to 70, or a fragment thereof; (c) polynucleotides comprising a nucleotide sequence capable of hybridising to the complement of the nucleotide sequences set out in Examples 1 to 70 or a fragment thereof; (d) polynucleotides comprising a polynucleotide sequence which is degenerate as a result of the genetic code to the polynucleotides defined in (a), (b) or (c).

There is provided, according to another aspect of the present invention, a polynucleotide selected from: (a) polynucleotides comprising any one of the nucleotide sequences set out in Examples 1 to 14 or the complement thereof; (b) polynucleotides comprising a nucleotide sequence capable of hybridising to the nucleotide sequences set out in Examples 1 to 14, or a fragment thereof; (c) polynucleotides comprising a nucleotide sequence capable of hybridising to the complement of the nucleotide sequences set out in Examples 1 to 14 or a fragment thereof; (d) polynucleotides comprising a polynucleotide sequence which is degenerate as a result of the genetic code to the polynucleotides defined in (a), (b) or (c).

We provide, according to yet a further aspect of the present invention, a polynucleotide selected from: (a) polynucleotides comprising any one of the nucleotide sequences set out in Examples 15 to 19 or the complement thereof; (b) polynucleotides comprising a nucleotide sequence capable of hybridising to the nucleotide sequences set out in Examples 15 to 19, or a fragment thereof; (c) polynucleotides comprising a nucleotide sequence capable of hybridising to the complement of the nucleotide sequences set out in Examples 15 to 19 or a fragment thereof; (d) polynucleotides comprising a polynucleotide sequence which is degenerate as a result of the genetic code to the polynucleotides defined in (a), (b) or (c).

As a further aspect of the present invention, there is provided a polynucleotide selected from: (a) polynucleotides comprising any one of the nucleotide sequences set out in Examples 20 to 30 or the complement thereof; (b) polynucleotides comprising a nucleotide sequence capable of hybridising to the nucleotide sequences set out in Examples 20 to 30, or a fragment thereof; (c) polynucleotides comprising a nucleotide sequence capable of hybridising to the complement of the nucleotide sequences set out in Examples 20 to 30 or a fragment thereof; (d) polynucleotides comprising a polynucleotide sequence which is degenerate as a result of the genetic code to the polynucleotides defined in (a), (b) or (c).

We provide, according to a yet further aspect of the present invention, a polynucleotide selected from: (a) polynucleotides comprising any one of the nucleotide sequences set out in Examples 31 to 53 or the complement thereof; (b) polynucleotides comprising a nucleotide sequence capable of hybridising to the nucleotide sequences set out in Examples 31 to 53, or a fragment thereof; (c) polynucleotides comprising a nucleotide sequence capable of hybridising to the complement of the nucleotide sequences set out in 31 to 53 or a fragment thereof; (d) polynucleotides comprising a polynucleotide sequence which is degenerate as a result of the genetic code to the polynucleotides defined in (a), (b) or (c).

The present invention, in a further aspect, provides a polynucleotide selected from: (a) polynucleotides comprising any one of the nucleotide sequences set out in 54 to 70 or the complement thereof; (b) polynucleotides comprising a nucleotide sequence capable of hybridising to the nucleotide sequences set out in 54 to 70, or a fragment thereof; (c) polynucleotides comprising a nucleotide sequence capable of hybridising to the complement of the nucleotide sequences set out in 54 to 70 or a fragment thereof; (d) polynucleotides comprising a polynucleotide sequence which is degenerate as a result of the genetic code to the polynucleotides defined in (a), (b) or (c).

A polynucleotide probe which comprises a fragment of at least 15 nucleotides of a polynucleotide according to any of the above aspects of the invention.

The present invention also provides a polypeptide which comprises any one of the amino acid sequences set out in Examples 1 to 70 or in any of Examples 1 to 14, Examples 15 to 19, Examples 20 to 30, Examples 31 to 53 and Examples 54 to 70, or a homologue, variant, derivative or fragment thereof.

Preferably the polypeptide is encoded by a cDNA sequence obtainable from a eukaryotic cDNA library, preferably a metazoan cDNA library (such as insect or mammalian) said DNA sequence comprising a DNA sequence being selectively detectable with a Drosophila nucleotide sequence as shown in any one of Examples 1 to 70.

The term “selectively detectable” means that the cDNA used as a probe is used under conditions where a target cDNA of the invention is found to hybridize to the probe at a level significantly above background. The background hybridization may occur because of other cDNAs present in the cDNA library. In this event background implies a level of signal generated by interaction between the probe and a non-specific cDNA member of the library which is less than 10 fold, preferably less than 100 fold as intense as the specific interaction observed with the target cDNA. The intensity of interaction may be measured, for example, by radiolabelling the probe, e.g. with ³²P. Suitable conditions may be found by reference to the Examples, as well as in the detailed description below.

A polynucleotide encoding a polypeptide of the invention is also provided.

The present invention further provides a vector comprising a polynucleotide of the invention, for example an expression vector comprising a polynucleotide of the invention operably linked to a regulatory sequence capable of directing expression of said polynucleotide in a host cell.

Also provided is an antibody capable of binding a polypeptide of the invention.

In a further aspect the present invention provides a method for detecting the presence or absence of a polynucleotide of the invention in a biological sample which method comprises: (a) bringing the biological sample containing DNA or RNA into contact with a probe comprising a nucleotide of the invention under hybridising conditions; and (b) detecting any duplex formed between the probe and nucleic acid in the sample.

In another aspect the invention provides a method for detecting a polypeptide of the invention present in a biological sample which comprises: (a) providing an antibody of the invention; (b) incubating a biological sample with said antibody under conditions which allow for the formation of an antibody-antigen complex; and (c) determining whether antibody-antigen complex comprising said antibody is formed.

Knowledge of the genes involved in cell cycle progression allows the development of therapeutic agents for the treatment of medical conditions associated with aberrant cell cycle progression. Accordingly, the present invention provides a polynucleotide of the invention for use in therapy. The present invention also provides a polypeptide of the invention for use in therapy. The present invention further provides an antibody of the invention for use in therapy.

In a specific embodiment, the present invention provides a method of treating a tumour or a patient suffering from a proliferative disease, comprising administering to a patient in need of treatment an effective amount of a polynucleotide, polypeptide and/or antibody of the invention.

The present invention also provides the use of a polypeptide of the invention in a method of identifying a substance capable of affecting the function of the corresponding gene. For example, in one embodiment the present invention provides the use of a polypeptide of the invention in an assay for identifying a substance capable of inhibiting cell cycle progression. The substance may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. For example, possible functions of genes of the invention for which it may be desired to identify substances which affect such functions include chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In a further aspect the present invention provides a method for identifying a substance capable of binding to a polypeptide of the invention, which method comprises incubating the polypeptide with a candidate substance under suitable conditions and determining whether the substance binds to the polypeptide.

In an additional aspect, the invention provides kits comprising polynucleotides, polypeptides or antibodies of the invention and methods of using such kits in diagnosing the presence of absence of polynucleotides and polypeptides of the invention including deleterious mutant forms.

Also provided is a substance identified by the above methods of the invention. Such substances may be used in a method of therapy, such as in a method of affecting cell cycle progression, for example mitosis and/or meiosis.

The invention also provides a process comprising the steps of: (a) performing one of the above methods; and (b) preparing a quantity of those one or more substances identified as being capable of binding to a polypeptide of the invention.

Also provided is a process comprising the steps of: (a) performing one of the above methods; and (b) preparing a pharmaceutical composition comprising one or more substances identified as being capable of binding to a polypeptide of the invention.

We further provide a method for identifying a substance capable of modulating the function of a polypeptide of the invention or a polypeptide encoded by a polynucleotide of the invention, the method comprising the steps of: incubating the polypeptide with a candidate substance and determining whether activity of the polypeptide is thereby modulated.

A substance identified by a method or assay according to any of the above methods or processes is also provided, as is the use of such a substance in a method of inhibiting the function of a polypeptide. Use of such a substance in a method of regulating a cell division cycle function is also provided.

### BRIEF DESCRIPTION OF THE FIGURES

FIG. 1 shows a Facs analysis of propidium iodide staining after RNAi knockdown of pebble (in green) compared to control RNAi (RFP, in purple) in Dmel2 Drosophila

FIG. 2 shows a printout from a BLASTX comparison between Drosophila pebble, identified in Example 6 as a cell cycle gene, and human Ect2 (AK023067).

FIG. 3 shows a human Ect2 sequence, together with regions selected for designing two synthetic double stranded RNAs (COD1512 and COD1513) for siRNA knockdowns.

FIG. 4 shows a Western blot showing reduction of protein levels in 293 cells 72 hours after addition of siRNAs.

FIG. 5 is a graph showing FACs analysis of DNA content indicating cell cycle compartment of cells treated with siRNA for 48 hours.

FIG. 6 are fluorescence micrographs showing the effect of Ect2 siRNAi in U2OS cells. A multinucleate cells, B and C binuclear cell, D tri polar spindle with lagging chromatin.

FIG. 7 are fluorescence micrographs showing the effect of Ect2 siRNAi in Hela cells. Right: Multinicleate cells.

FIG. 8 shows Ect2 expression in tumour cells.

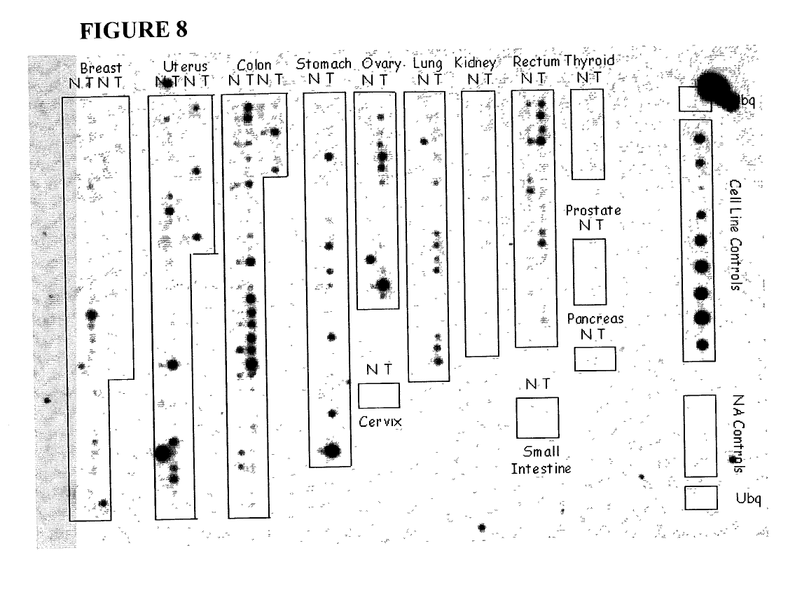

FIG. 9 shows expression of 6-His tagged human Ect2 protein in sf9 insect (baculovirus infected) cells.

### DETAILED DESCRIPTION OF THE INVENTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Preferably, the polypeptides and polynucleotides of the invention are such that they give rise to or are associated with defined phenotypes when mutated.

For example, mutations in the polypeptides and polynucleotides of the invention may be associated with a failure to complete cytokinesis; such polypeptides and polynucleotides are conveniently categorised as “Category 1”. Phenotypes associated with Category 1 polypeptides and polynucleotides include any one or more of the following, singly or in combination: Mitotic defects in brain: cytokinesis defect (polyploidy); Male semi-sterile, Meiotic defects in testis: cytokinesis defects, segregation defects. (Seg-01/62); Meiotic defects in testis: cytokinesis defects, abnormal spindles. (Ab-02/12); Mitotic defects in brain: cytokinesis defect (no overcondensation of diploids, high polyploidy); Meiotic defects in testis: cytokinesis defects. Dark bands in eyes, dominant; Meiotic defects in testis: cytokinesis defects; Meiotic defects in testis:segregation defect, cytokinesis defect (Ck-09/32); Mitotic defects in brain: cytokinesis defect (no overcondensation of diploids, very high polyploidy); Mitotic defects in brain: cytokinesis defect (very high polyploidy); Mitotic defects in brain: cytokinesis defect. Meiotic defects in testis: cytokinesis defects (Mitotic higher level of condensation, polyploidy, Meiotic: Ck05/07); Mitotic defects in brain, Cytokinesis defect (no overcondensation of diploids, high polyploidy); Mitotic defects in brain: cytokinesis defect (very high polyploidy, chromosomes entangled?); Mitotic defects in brain: cytokinesis defect (very high polyploidy; Meiotic defects in testis: cytokinesis defects (Ck-04/06); Female sterile (anaphase bridges, lagging chromosomes); Mitotic defects in brain: cytokinesis defect. Meiotic defects in testis: cytokinesis defects:(mitotic: high polyploidy, no diploids, higher mitotic index, meiotic: Ck-01/05); Meiotic defects in testis: cytokinesis defects; Meiotic defects in testis: cytokinesis defects(Ck-06/09); Meiotic defects in testis: segregation defects, cytokinesis defect(Ck-07/35); Meiotic defects in testis: cytokinesis defects.

Alternatively, mutations in the polypeptides and polynucleotides of the invention may be associated with a failure to enter M-phase; such polypeptides and polynucleotides are conveniently categorised as “Category 2”. Phenotypes associated with Category 2 polypeptides and polynucleotides include any one or more of the following, singly or in combination: Meiotic defects in testis: no division (no meiosis); Mitotic defects in brain: no mitosis; Meiotic defects in testis: segregation defects, meiotic failure (Mf-07/75); Meiotic defects in testis: segregation defects, meiotic failure (Mf-05/31); Meiotic defects in testis: cytokinesis defects, meiotic failure (Mf-02/15).

Mutations in the polypeptides and polynucleotides of the invention may be associated with a metaphase arrest phenotype (“Category 3”). Phenotypes associated with Category 3 polypeptides and polynucleotides include any one or more of the following, singly or in combination: Mitotic defects in brain: prometaphase arrest (overcondensation, polyploidy, scattered chromosomes with bipolar spindle); Male sterile, Female sterile, Mitotic defects in brain: prometaphase arrest (Overcondensation, polyploidy, fewer anaphases, high mitotic index, scattered chromosomes with bipolar spindle); Mitotic defects in brain: (weak overcondensation, metaphase with bipolar spindle); Mitotic defects in brain: prometaphase arrest; Mitotic defects in brain: metaphase arrest; Mitotic defects in brain: metaphase arrest. (overcondensation, polyploidy, aneuploidy, few anaphases, high mitotic index, metaphase with bent bipolar spindle); Mitotic defects in brain: metaphase arrest. (overcondensation, polyploidy, few anaphases, high mitotic index, metaphase with bent bipolar spindle); Mitotic defects in brain: Metaphase arrest (overcondensation, polyploidy, aneuploidy, no anaphases, high mitotic index, metaphase with bipolar spindle); Mitotic defects in brain: metaphase arrest (overcondensation, metaphase with bipolar spindle; Meiotic defects in testis: segregation defects, multipolar spindles (Mul-02/29); Meiotic defects in testis: cytokinesis defects, abnormal spindles. (Ab-01/03); Mitotic defects in brain: metaphase arrest; Mitotic defects in brain: metaphase arrest (overcondensation, polyploidy, metaphase with bipolar spindle); Mitotic defects in brain: metaphase arrest. Meiotic defects in testis: segregation defects. Abnormal spindles (mitotic: High mitotic index, meiotic: Ab-08/24); Mitotic defects in brain: metaphase arrest (overcondensation, few anaphases, some polyploids); Mitotic defects in brain: prometaphase arrest (overcondensation, fewer anaphases, metaphase with bipolar spindle); Mitotic defects in brain: metaphase arrest (condensation, no polyploidy, no anaphases, metaphase with bipolar spindle).

Mutations in Category 4 polypeptides and polynucleotides of the invention may be associated with an anaphase defect phenotype; phenotypes associated with Category 4 polypeptides and polynucleotides include any one or more of the following, singly or in combination: Mitotic defects in brain: anaphase defects (overcondensation, high polyploidy, some lagging chromosomes); Meiotic defects in testis: segregation defects; Male and female sterile, small wings, meiotic defects in testis: segregation defects, elongation defect; Mitotic defects in brain: anaphase defects (overcondensation, anaphase bridge, metaphase with swollen chromosomes and bipolar spindle); Mitotic defects in brain: Anaphase defects. (overcondensation, aneuploidy, some lagging chromosomes and breaks); Meiotic defects in testis: segregation defects; Meiotic defects in testis: segregation defects, multi-stage defects (Pl-02/17); Meiotic defects in testis: segregation defects, multi-stage defects (Pl-02/18); Meiotic defects in testis: cytokinesis defects, segregation defects (seg-01/01); Mitotic defects in brain: cytokinesis defect. Meiotic defects in testis: cytokinesis defect. Multi-stage defects Polyploidy, no overcondensation Pl-01/10; Meiotic defects in testis: segregation defects, abnormal spindles. (Ab-03/30); Mitotic defects in brain: anaphase defects (weak, higher condensation, some polyploidy, fewer anaphases, polyploids with monopolar spindles); Mitotic defects in brain: anaphase defects (overcondensation, polyplody (with overcondensation), few anaphases, metaphase with bipolar spindle); Meiotic defects in testis: cytokinesis defects; Meiotic defects in testis: segregation defects, multipolar spindles(Mul-02/22); Meiotic defects in testis: segregation defects, abnormal spindles (Ab-04/26); Meiotic defects in testis: cytokinesis defects, abnormal spindles (Ab-16/13); Mitotic defects in brain: anaphase defects. Meiotic defects in testis: segregation defects, abnormal spindles (mitotic: Overcondensation, lagging chromosomes/less aligned metaphase with bipolar spindles, Meiotic: Ab-06/20); Meiotic defects in testis: segregation defects; Meiotic defects in testis: no division (no meiosis); Meiotic defects in testis: segregation defects, abnormal spindles (Ab-12/48); Meiotic defects in testis: segregation defects, multipolar spindles (mitotic: High polyploids, no diploids, higher mitotic index Meiotic: Mul-02/59); Meiotic defects in testis: segregation defect; Meiotic defects in testis: segregation defects, abnormal spindles (meiotic: Ab-08/42); Female sterile. Meiotic defects in testis: cytokinesis defects, segregation defects (Mitotic: Less condensed chromosomes, nuclear bridges, Meiotic: Seg-01/02; Mitotic defects in brain: anaphase defects; Meiotic defects in testis: cytokinesis defects, abnormal spindles(Ab-01/04); Meiotic defects in testis: segregation defects (overcondensation, fewer anaphases); Mitotic defects in brain:(some overcondensation, anaphase bridge, metaphase with swollen chromosome and bipolar spindle).

A fifth category (“Category 5”) of polypeptides and polynucleotides of the invention are associated with the presence of small imaginal discs (block to proliferation). Phenotypes associated with Category 5 polypeptides and polynucleotides include any one or more of the following, singly or in combination: 2nd chromosome, small imaginal discs.

The polypeptides and polynucleotides of the invention may also be categorised according to their function, or their putative function.

For example, the polypeptides described here preferably comprise, and the polynucleotides described here are ones which preferably encode polypeptides comprising, any one or more of the following: a CBP activator protein; a CCR4-associated regulator of polymerase II transcription; a CTP synthase (CTPS); a Cyclin specific ubiquitin conjugating enzyme; a DNA packaging protein; a DNA repair protein; a DNA-binding protein involved in chromosomal organisation; a DNase IV; a EIF4G2 translation initiation factor; a eukaryotic translation initiation factor 6; a Ecdysone-induced protein 78C; a Egf2 translation factor; a G protein-coupled receptor kinase 7; a GTPase exchange factor; a phosphatidylinositol transfer protein beta isoform; a His-rich protein; a Lk6 kinase; a MAP kinase; a MAP kinase interacting kinase 1; a N-arginine dibasic convertase; a Phosphatidylinositol transfer protein; a RIP protein kinase; a RNA binding motif, single stranded interacting protein; a RNA binding protein; a RYKreceptor tyrosine kinase; a Ribosomal protein L1; a selenide,water dikinase 1; a selenium donor protein 1; a selenophosphate synthetase 1; a Sqv-7-like protein; a sugar modification protein; a protein involved in cytokinesis and signalling; a TEK tyrosine kinase; a Translation elongation factor; a UDP-galactose transporter; a v-erba related protein; a WD40 protein; a brahma protein; a calcium binding protein; a cell adhesion protein; a chaperone; a chromodomain helicase DNA binding protein; a chromodomain-helicase-DNA-binding protein; a coiled coil protein with ubiquitin like domain; a component of the 19S proteasome regulatory particle; a couch potato RNA binding protein; a cytidine 5-prime triphosphate synthetasea; a cytoskeletal structural protein; a death domain containing protein; a developmentally expressed in axons of the CNS; a diacylglycerol-activated/phosholipid dependent protein kinase C inhibitor; a diazepam binding inhibitor; a diphosphate kinase; a dodecasattelite DNA binding protein; a doughnut protein tyrosine kinase; an elongation factor 2; a endoplasmic reticulum ATPase; a eukaryotic translation initiation factor 4E binding protein 2; a factor involved in axon guidance; a fatty-acid-Coenzyme A ligase; a flap structure-specific endonuclease 1; a protein involved in the formation of the contractile ring and the initiation of cytokinesis; a glucose-6-phosphate transporter; a glycoprotein glucosyltransferase; a growth factor; a transmembrane receptor protein tyrosine kinase involved in cell growth and maintenance; a guanyl-nucleotide exchange factor involved in signal transduction; a heat shock protein; a helicase; a high density lipoprotein binding protein; a histone acetyl transferase transcriptional activator; a histone acetyltransferase; a histone acetyltransferase GCN5; a protein involved in development of the abdomen (embryos); a protein involved in the development of the imaginal discs (larvae or pupae); a kinesin like protein 67a; a ligand-dependent nuclear receptor; a ligand-dependent nuclear receptor; a lola-like specific RNA polymerase II transcription factor; a matrix associated protein; a membrane glycoprotein; a mitotic heterochromatin fragment clone CH(2)6; a motor protein; a motor protein involved in cytoskeleton organization; a mushroom body RNA binding protein; a myosin like proteins; a nemo-like kinase; a non-ATPase protein; a nuclear receptor NR1E1; a nucleic acid binding protein; a nucleoside diphosphate kinase (NBR-A); a oly(rC)-binding protein 2 (hnRNP-E1); a peroxisome biogenesis factor 1; a phosopholipid transporter involved in lipid metabolism; a phosphatase or enhancer of Pi uptake protein; a protease; a proteasome regulatory particle; a protein involved in cytoskeleton organization and/or biogenesis; a protein kinase associated with microtubules; a protein kinase mitogen-activated 7; a protein serine/threonine kinase involved in cell cycle, possibly targeted to cytoskeleton; a protein serine/threonine kinase involved in eye morphogenesis; a protein which associates with cdc25 phosphatase; a protein which induces apoptosis; a ribonuclease P; a ribonuclease P protein subunit p29; a ser/thr phosphatase; a signal transduction protein; a signal transport protein; a sin3-associated polypeptide; a single stranded DNA/RNA binding protein; a sodium-dependent dicarboxylate transporters; a ssDNA/RNA binding proteins; a striatin, calmodulin-binding protein (STRN); a structural protein of ribosome involved in protein biosynthesis; a subtelomeric heterochromatin repeats; a sugar acetylase; a sugar modification protein; a suppresspr of ras; a tRNA processing enzyme Ribonuclease P protein subunit; a thyroid hormone responsive gene; a tie receptor protein tyrosine kinase; a transacylase; a transcription factor; a transcription factor involved in chromatin remodelling; a transcriptional regulation of c-myc expression; a transcriptional regulator; a transcriptional regulators/telomeric silencing; a translation initiation factor; a tumor metastasis inhibitor; a tyrosine 3-monooxygenase/tryptophan 5-monooxygenase activation protein; a ubiquitin carrier protein; a ubiquitin-conjugating enzyme; a ugtUDP-glucose-glycoprotein glucosyltransferase; a zinc finger protein; an RNA polymerase II transcription factor; an acetylcholinesterase (YT blood group) precursor; an actin binding protein; an actin dependent regulator of chromatin; an acyl-CoA-binding protein; an alanine:glyoxylate aminotransferase; an alpha esterase; an ankyrin protein; an imitation-SWI protein; and an integrin beta 4 binding protein.

Polypeptides

It will be understood that polypeptides of the invention are not limited to polypeptides having the amino acid sequence set out in Examples 1 to 70 or fragments thereof but also include homologous sequences obtained from any source, for example related viral/bacterial proteins, cellular homologues and synthetic peptides, as well as variants or derivatives thereof.

Thus polypeptides of the invention also include those encoding homologues from other species including animals such as mammals (e.g. mice, rats or rabbits), especially primates, more especially humans. More specifically, homologues included within the scope of the invention include human homologues.

Thus, the present invention covers variants, homologues or derivatives of the amino acid sequence set out in Examples 1 to 70, as well as variants, homologues or derivatives of the nucleotide sequence coding for the amino acid sequences of the present invention.

In the context of the present invention, a homologous sequence is taken to include an amino acid sequence which is at least 15, 20, 25, 30, 40, 50, 60, 70, 80 or 90% identical, preferably at least 95 or 98% identical at the amino acid level over at least 50 or 100, preferably 200, 300, 400 or 500 amino acids with any one of the polypeptide sequences shown in the Examples. In particular, homology should typically be considered with respect to those regions of the sequence known to be essential for protein function rather than non-essential neighbouring sequences. This is especially important when considering homologous sequences from distantly related organisms.

Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These publicly and commercially available computer programs can calculate % homology between two or more sequences.

% homology may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The terms “variant” or “derivative” in relation to the amino acid sequences of the present invention includes any substitution of, variation of, modification of, replacement of, deletion of or addition of one (or more) amino acids from or to the sequence providing the resultant amino acid sequence retains substantially the same activity as the unmodified sequence, preferably having at least the same activity as the polypeptides presented in the sequence listings in the Examples.

Polypeptides having the amino acid sequence shown in the Examples, or fragments or homologues thereof may be modified for use in the present invention. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides of the invention. Amino acid substitutions may include the use of non-naturally occurring analogues, for example to increase blood plasma half-life of a therapeutically administered polypeptide.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Polypeptides of the invention also include fragments of the full length sequences mentioned above. Preferably said fragments comprise at least one epitope. Methods of identifying epitopes are well known in the art. Fragments will typically comprise at least 6 amino acids, more preferably at least 10, 20, 30, 50 or 100 amino acids.

Proteins of the invention are typically made by recombinant means, for example as described below. However they may also be made by synthetic means using techniques well known to skilled persons such as solid phase synthesis. Proteins of the invention may also be produced as fusion proteins, for example to aid in extraction and purification. Examples of fusion protein partners include glutathione-S-transferase (GST), 6xHis, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the function of the protein of interest sequence. Proteins of the invention may also be obtained by purification of cell extracts from animal cells.

Proteins of the invention may be in a substantially isolated form. It will be understood that the protein may be mixed with carriers or diluents which will not interfere with the intended purpose of the protein and still be regarded as substantially isolated. A protein of the invention may also be in a substantially purified form, in which case it will generally comprise the protein in a preparation in which more than 90%, e.g. 95%, 98% or 99% of the protein in the preparation is a protein of the invention.

A polypeptide of the invention may be labeled with a revealing label. The revealing label may be any suitable label which allows the polypeptide to be detected. Suitable labels include radioisotopes, e.g. ¹²⁵I, enzymes, antibodies, polynucleotides and linkers such as biotin. Labeled polypeptides of the invention may be used in diagnostic procedures such as immunoassays to determine the amount of a polypeptide of the invention in a sample. Polypeptides or labeled polypeptides of the invention may also be used in serological or cell-mediated immune assays for the detection of immune reactivity to said polypeptides in animals and humans using standard protocols.

A polypeptide or labeled polypeptide of the invention or fragment thereof may also be fixed to a solid phase, for example the surface of an immunoassay well or dipstick. Such labeled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

Polypeptides of the invention may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides of the invention may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products of the invention. Such cell culture systems in which polypeptides of the invention are expressed may be used in assay systems to identify candidate substances which interfere with or enhance the functions of the polypeptides of the invention in the cell.

Polynucleotides

Polynucleotides of the invention include polynucleotides that comprise any one or more of the nucleic acid sequences set out in Examples 1 to 70 and fragments thereof. Polynucleotides of the invention also include polynucleotides encoding the polypeptides of the invention. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

Polynucleotides of the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

The terms “variant”, “homologue” or “derivative” in relation to the nucleotide sequence of the present invention include any substitution of, variation of, modification of, replacement of, deletion of or addition of one (or more) nucleic acid from or to the sequence. Preferably said variant, homologues or derivatives code for a polypeptide having biological activity.

As indicated above, with respect to sequence homology, preferably there is at least 50 or 75%, more preferably at least 85%, more preferably at least 90% homology to the sequences shown in the sequence listing herein. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides of the invention capable of selectively hybridising to the nucleotide sequences presented herein, or to their complement, will be generally at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” means that the polynucleotide used as a probe is used under conditions where a target polynucleotide of the invention is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0).

Where the polynucleotide of the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the Examples. Such sequences may be obtained by probing cDNA libraries made from or genomic DNA libraries from other animal species, and probing such libraries with probes comprising all or part of any on of the sequences shown in the Examples under conditions of medium to high stringency. The nucleotide sequences of the human homologues described in the Examples, may preferably be used to identify other primate/mammalian homologues since nucleotide homology between human sequences and mammalian sequences is likely to be higher than is the case for the Drosophila sequences identified herein.

Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labeled fragments the sequences disclosed in the Examples.

In addition, homologous sequences may be identified by searching nucleotide and/or protein databases using search algorithms such as the BLAST suite of programs. This approach is described in the Examples.

Alternatively, such polynucleotides may be obtained by site directed mutagenesis of characterised sequences, such as the sequences disclosed in the Examples. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. For example, further changes may be desirable to represent particular coding changes found in the sequences disclosed in the Examples which give rise to mutant genes which have lost their regulatory function. Probes based on such changes can be used as diagnostic probes to detect such mutants.

Polynucleotides of the invention may be used to produce a primer, e.g. a PCR primer, a primer for an alternative amplification reaction, a probe e.g. labeled with a revealing label by conventional means using radioactive or non-radioactive labels, or the polynucleotides may be cloned into vectors. Such primers, probes and other fragments will be at least 8, 9, 10, or 15, preferably at least 20, for example at least 25, 30 or 40 nucleotides in length, and are also encompassed by the term polynucleotides of the invention as used herein.

Polynucleotides such as a DNA polynucleotides and probes according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Polynucleotides or primers of the invention may carry a revealing label. Suitable labels include radioisotopes such as ³²P or ³⁵S, enzyme labels, or other protein labels such as biotin. Such labels may be added to polynucleotides or primers of the invention and may be detected using by techniques known per se.

Polynucleotides or primers of the invention or fragments thereof labeled or unlabeled may be used by a person skilled in the art in nucleic acid-based tests for detecting or sequencing polynucleotides of the invention in the human or animal body.

Such tests for detecting generally comprise bringing a biological sample containing DNA or RNA into contact with a probe comprising a polynucleotide or primer of the invention under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing nucleotides of the invention include bringing a biological sample containing target DNA or RNA into contact with a probe comprising a polynucleotide or primer of the invention under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

Tests for detecting or sequencing nucleotides of the invention in a biological sample may be used to determine particular sequences within cells in individuals who have, or are suspected to have, an altered gene sequence, for example within cancer cells including leukaemia cells and solid tumours such as breast, ovary, lung, colon, pancreas, testes, liver, brain, muscle and bone tumours. Cells from patients suffering from a proliferative disease may also be tested in the same way.

In addition, the identification of the genes described in the Examples will allow the role of these genes in hereditary diseases to be investigated. In general, this will involve establishing the status of the gene (e.g. using PCR sequence analysis), in cells derived from animals or humans with, for example, neurological disorders or neoplasms.

The probes of the invention may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like.

Nucleic Acid Vectors

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells include bacteria such as E. coli, yeast, mammalian cell lines and other eukaryotic cell lines, for example insect Sf9 cells.

Preferably, a polynucleotide of the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors of the invention may be transformed or transfected into a suitable host cell as described below to provide for expression of a protein of the invention. This process may comprise culturing a host cell transformed with an expression vector as described above under conditions to provide for expression by the vector of a coding sequence encoding the protein, and optionally recovering the expressed protein. Vectors will be chosen that are compatible with the host cell used.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the polypeptide of the invention include promoters/enhancers and other expression regulation signals. These control sequences may be selected to be compatible with the host cell for which the expression vector is designed to be used in. The term promoter is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells, such as insect cells, may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

Polynucleotides according to the invention may also be inserted into the vectors described above in an antisense orientation to provide for the production of antisense RNA. Antisense RNA or other antisense polynucleotides may also be produced by synthetic means. Such antisense polynucleotides may be used in a method of controlling the levels of RNAs transcribed from genes comprising any one of the polynucleotides of the invention.

Host Cells

Vectors and polynucleotides of the invention may be introduced into host cells for the purpose of replicating the vectors/polynucleotides and/or expressing the polypeptides of the invention encoded by the polynucleotides of the invention. Although the polypeptides of the invention may be produced using prokaryotic cells as host cells, it is preferred to use eukaryotic cells, for example yeast, insect or mammalian cells, in particular mammalian cells. Vectors/polynucleotides of the invention may be introduced into suitable host cells using a variety of techniques known in the art, such as transfection, transformation and electroporation. Where vectors/polynucleotides of the invention are to be administered to animals, several techniques are known in the art, for example infection with recombinant viral vectors such as retroviruses, herpes simplex viruses and adenoviruses, direct injection of nucleic acids and biolistic transformation.

Protein Expression and Purification

Host cells comprising polynucleotides of the invention may be used to express polypeptides of the invention. Host cells may be cultured under suitable conditions which allow expression of the proteins of the invention. Expression of the polypeptides of the invention may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

Polypeptides of the invention can be extracted from host cells by a variety of techniques known in the art, including enzymatic, chemical and/or osmotic lysis and physical disruption.

Polypeptides of the invention may also be produced recombinantly in an in vitro cell-free system, such as the TnT™ (Promega) rabbit reticulocyte system.

Antibodies

The invention also provides monoclonal or polyclonal antibodies to polypeptides of the invention or fragments thereof. Thus, the present invention further provides a process for the production of monoclonal or polyclonal antibodies to polypeptides of the invention.

If polyclonal antibodies are desired, a selected mammal (e.g., mouse, rabbit, :goat, horse, etc.) is immunised with an immunogenic polypeptide bearing an epitope(s) from a polypeptide of the invention. Serum from the immunised animal is collected and treated according to known procedures. If serum containing polyclonal antibodies to an epitope from a polypeptide of the invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes in the polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes in the polypeptides of the invention can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against epitopes from polypeptides of the invention are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the agent against which protection is desired.

Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Antibodies may be used in method of detecting polypeptides of the invention present in biological samples by a method which comprises: (a) providing an antibody of the invention; (b) incubating a biological sample with said antibody under conditions which allow for the formation of an antibody-antigen complex; and (c) determining whether antibody-antigen complex comprising said antibody is formed.

Suitable samples include extracts tissues such as brain, breast, ovary, lung, colon, pancreas, testes, liver, muscle and bone tissues or from neoplastic growths derived from such tissues.

Antibodies of the invention may be bound to a solid support and/or packaged into kits in a suitable container along with suitable reagents, controls, instructions and the like.

Assays

The present invention provides assays that are suitable for identifying substances which bind to polypeptides of the invention and which affect, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, cytokinesis functions, chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In addition, assays suitable for identifying substances that interfere with binding of polypeptides of the invention, where appropriate, to components of cell division cycle machinery. This includes not only components such as microtubules but also signalling components and regulatory components as indicated above. Such assays are typically in vitro. Assays are also provided that test the effects of candidate substances identified in preliminary in vitro assays on intact cells in whole cell assays. The assays described below, or any suitable assay as known in the art, may be used to identify these substances.

According to one aspect of the invention, therefore, we provide one or more substances identified by any of the assays described below, viz, mitosis assays, meiotic assays, polypeptide binding assays, microtubule binding/polymerisation assays, microtubule purification and binding assays, microtubule organising centre (MTOC) nucleation activity assays, motor protein assay, assay for spindle assembly and function, assays for dna replication, chromosome condensation assays, kinase assays, kinase inhibitor assays, and whole cell assays, each as described in further detail below.

Candidate Substances

A substance that inhibits cell cycle progression as a result of an interaction with a polypeptide of the invention may do so in several ways. For example, if the substance inhibits cell division, mitosis and/or meiosis, it may directly disrupt the binding of a polypeptide of the invention to a component of the spindle apparatus by, for example, binding to the polypeptide and masking or altering the site of interaction with the other component. A substance which inhibits DNA replication may do so by inhibiting the phosphorylation or de-phosphorylation of proteins involved in replication. For example, it is known that the kinase inhibitor 6-DMAP (6-dimethylaminopurine) prevents the initiation of replication (Blow, J J, 1993, J Cell Biol122,993-1002). Candidate substances of this type may conveniently be preliminarily screened by in vitro binding assays as, for example, described below and then tested, for example in a whole cell assay as described below. Examples of candidate substances include antibodies which recognise a polypeptide of the invention.

A substance which can bind directly to a polypeptide of the invention may also inhibit its function in cell cycle progression by altering its subcellular localisation and hence its ability to interact with its normal substrate. The substance may alter the subcellular localisation of the polypeptide by directly binding to it, or by indirectly disrupting the interaction of the polypeptide with another component. For example, it is known that interaction between the p68 and p180 subunits of DNA polymerase alpha-primase enzyme is necessary in order for p180 to translocate into the nucleus (Mizuno et al (1998) Mol Cell Biol18,3552-62), and accordingly, a substance which disrupts the interaction between p68 and p180 will affect nuclear translocation and hence activity of the primase. A substance which affects mitosis may do so by preventing the polypeptide and components of the mitotic apparatus from coming into contact within the cell. These substances may be tested using, for example the whole cells assays described below. Non-functional homologues of a polypeptide of the invention may also be tested for inhibition of cell cycle progression since they may compete with the wild type protein for binding to components of the cell division cycle machinery whilst being incapable of the normal functions of the protein or block the function of the protein bound to the cell division cycle machinery. Such non-functional homologues may include naturally occurring mutants and modified sequences or fragments thereof.

Alternatively, instead of preventing the association of the components directly, the substance may suppress the biologically available amount of a polypeptide of the invention. This may be by inhibiting expression of the component, for example at the level of transcription, transcript stability, translation or post-translational stability. An example of such a substance would be antisense RNA or double-stranded interfering RNA sequences which suppresses the amount of mRNA biosynthesis.

Suitable candidate substances include peptides, especially of from about 5 to 30 or 10 to 25 amino acids in size, based on the sequence of the polypeptides described in the Examples, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for a polypeptide of the invention. Furthermore, combinatorial libraries, peptide and peptide mimetics, defined chemical entities, oligonucleotides, and natural product libraries may be screened for activity as inhibitors of binding of a polypeptide of the invention to the cell division cycle machinery, for example mitotic/meiotic apparatus (such as microtubules). The candidate substances may be used in an initial screen in batches of, for example 10 substances per reaction, and the substances of those batches which show inhibition tested individually. Candidate substances which show activity in in vitro screens such as those described below can then be tested in whole cell systems, such as mammalian cells which will be exposed to the inhibitor and tested for inhibition of any of the stages of the cell cycle.

Polypeptide Binding Assays

One type of assay for identifying substances that bind to a polypeptide of the invention involves contacting a polypeptide of the invention, which is immobilised on a solid support, with a non-immobilised candidate substance determining whether and/or to what extent the polypeptide of the invention and candidate substance bind to each other. Alternatively, the candidate substance may be immobilised and the polypeptide of the invention non-immobilised.

In a preferred assay method, the polypeptide of the invention is immobilised on beads such as agarose beads. Typically this is achieved by expressing the component as a GST-fusion protein in bacteria, yeast or higher eukaryotic cell lines and purifying the GST-fusion protein from crude cell extracts using glutathione-agarose beads (Smith and Johnson, 1988). As a control, binding of the candidate substance, which is not a GST-fusion protein, to the immobilised polypeptide of the invention is determined in the absence of the polypeptide of the invention. The binding of the candidate substance to the immobilised polypeptide of the invention is then determined. This type of assay is known in the art as a GST pulldown assay. Again, the candidate substance may be immobilised and the polypeptide of the invention non-immobilised.

It is also possible to perform this type of assay using different affinity purification systems for immobilising one of the components, for example Ni-NTA agarose and histidine-tagged components.

Binding of the polypeptide of the invention to the candidate substance may be determined by a variety of methods well-known in the art. For example, the non-immobolised component may be labeled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Microtubule Binding/Polymerisation Assays

In the case of polypeptides of the invention that bind to microtubules, another type of in vitro assay involves determining whether a candidate substance modulates binding of a polypeptide of the invention to microtubules. Such an assay typically comprises contacting a polypeptide of the invention with microtubules in the presence or absence of the candidate substance and determining if the candidate substance has an affect on the binding of the polypeptide of the invention to the microtubules. This assay can also be used in the absence of candidate substances to confirm that a polypeptide of the invention does indeed bind to microtubules. Microtubules may be prepared and assays conducted as follows:

Microtubule Purification and Binding Assays

Microtubules are purified from 0-3h-old Drosophila embryos essentially as described previously (Saunders, et al., 1997). About 3 ml of embryos are homogenized with a Dounce homogenizer in 2 volumes of ice-cold lysis buffer (0.1 M Pipes/NaOH, pH6.6, 5 mM EGTA, 1 mM MgSO4, 0.9 M glycerol, 1 mM DTT, 1 mM PMSF, 1 μg/ml aprotinin, 1 μg/ml leupeptin and 1 μg/ml pepstatin). The microtubules are depolymerized by incubation on ice for 15 min, and the extract is then centrifuged at 16,000 g for 30 min at 4° C. The supernatant is recentrifuged at 135,000 g for 90 min at 4° C. Microtubules in this later supernatant are polymerized by addition of GTP to 1 mM and taxol to 20 μM and incubation at room temperature for 30 min. A 3 ml aliquot of the extract is layered on top of 3 ml 15% sucrose cushion prepared in lysis buffer. After centrifuging at 54,000 g for 30 min at 20° C. using a swing out rotor, the microtubule pellet is resuspended in lysis buffer.

Microtubule overlay assays are performed as previously described (Saunders et al., 1997). 500 ng per lane of recombinant Asp, recombinant polypeptide, and bovine serum albumin (BSA, Sigma) are fractionated by 10% SDS-PAGE and blotted onto PVDF membranes (Millipore). The membranes are preincubated in TBST (50 mM Tris pH 7.5, 150 mM NaCl, 0.05% Tween 20) containing 5% low fat powdered milk (LFPM) for 1 h and then washed 3 times for 15 min in lysis buffer. The filters are then incubated for 30 minutes in lysis buffer containing either 1 mM GDP, 1 mM GTP, or 1 mM GTP-γ-S. MAP-free bovine brain tubulin (Molecular Probes) is polymerised at a concentration of 2 μg/ml in lysis buffer by addition of GTP to a final concentration of 1 mM and incubated at 37° C. for 30 min. The nucleotide solutions are removed and the buffer containing polymerised microtubules added to the membanes for incubation for 1 h at 37° C. with addition of taxol at a final concentration of 10 μM for the final 30 min. The blots are then washed 3 times with TBST and the bound tubulin detected using standard Western blot procedures using anti-β-tubulin antibodies (Boehringer Manheim) at 2.5 μg/ml and the Super Signal detection system (Pierce).

It may be desirable in one embodiment of this type of assay to deplete the polypeptide of the invention from cell extracts used to produce polymerise microtubules. This may, for example, be achieved by the use of suitable antibodies.

A simple extension to this type of assay would be to test the effects of purified polypeptide of the invention upon the ability of tubulin to polymerise in vitro (for example, as used by Andersen and Karsenti, 1997) in the presence or absence of a candidate substance (typically added at the concentrations described above). Xenopus cell-free extracts may conveniently be used, for example as a source of tubulin.

Microtubule Organising Centre (MTOC) Nucleation Activity Assays

Candidate substances, for example those identified using the binding assays described above, may be screening using a microtubule organising centre nucleation activity assay to determine if they are capable of disrupting MTOCs as measured by, for example, aster formation. This assay in its simplest form comprises adding the candidate substance to a cellular extract which in the absence of the candidate substance has microtubule organising centre nucleation activity resulting in formation of asters.

In a preferred embodiment, the assay system comprises (i) a polypeptide of the invention and (ii) components required for microtubule organising centre nucleation activity except for functional polypeptide of the invention, which is typically removed by immunodepletion (or by the use of extracts from mutant cells). The components themselves are typically in two parts such that microtubule nucleation does not occur until the two parts are mixed. The polypeptide of the invention may be present in one of the two parts initially or added subsequently prior to mixing of the two parts.

Subsequently, the polypeptide of the invention and candidate substance are added to the component mix and microtubule nucleation from centrosomes measured, for example by immunostaining for the polypeptide of the invention and visualising aster formation by immuno-fluorescence microscopy. The polypeptide of the invention may be preincubated with the candidate substance before addition to the component mix. Alternatively, both the polypeptide of the invention and the candidate substance may be added directly to the component mix, simultaneously or sequentially in either order.

The components required for microtubule organising centre formation typically include salt-stripped centrosomes prepared as described in Moritz et al., 1998. Stripping centrosome preparations with 2 M KI removes the centrosome proteins CP60, CP190, CNN and γ-tubulin. Of these, neither CP60 nor CP190 appear to be required for microtubule nucleation. The other minimal components are typically provided as a depleted cellular extract, or conveniently, as a cellular extract from cells with a non-functional variant of a polypeptide of the invention. Typically, labeled tubulin (usually β-tubulin) is also added to assist in visualising aster formation.

Alternatively, partially purified centrosomes that have not been salt-stripped may be used as part of the components. In this case, only tubulin, preferably labeled tubulin is required to complete the component mix.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

The degree of inhibition of aster formation by the candidate substance may be determinedby measuring the number of normal asters per unit area for control untreated cell preparation and measuring the number of normal asters per unit area for cells treated with the candidate substance and comparing the result. Typically, a candidate substance is considered to be capable of disrupting MTOC integrity if the treated cell preparations have less than 50%, preferably less than 40, 30, 20 or 10% of the number of asters found in untreated cells preparations. It may also be desirable to stain cells for γ-tubulin to determine the maximum number of possible MTOCs present to allow normalisation between samples.

Motor Protein Assay

Polypeptides of the invention may interact with motor proteins such as the Eg5-like motor protein in vitro. The effects of candidate substances on such a process may be determined using assays wherein the motor protein is immobilised on coverslips. Rhodamine labeled microtubules are then added and their translocation can be followed by fluorescent microscopy. The effect of candidate substances may thus be determined by comparing the extent and/or rate of translocation in the presence and absence of the candidate substance. Generally, candidate substances known to bind to a polypeptide of the invention, would be tested in this assay. Alternatively, a high throughput assay may be used to identify modulators of motor proteins and the resulting identified substances tested for affects on a polypeptide of the invention as described above.

Typically this assay uses microtubules stabilised by taxol (e.g. Howard and Hyman 1993; Chandra and Endow, 1993—both chapters in “Motility Assays for Motor Proteins” Ed Jon Scholey, pub Academic Press). If however, a polypeptide of the invention were to promote stable polymerisation of microtubules (see above) then these microtubules could be used directly in motility assays.

Simple protein-protein binding assays as described above, using a motor protein and a polypeptide of the invention may also be used to confirm that the polypeptide of the invention binds to the motor protein, typically prior to testing the effect of candidate substances on that interaction.

Assay for Spindle Assembly and Function

A further assay to investigate the function of polypeptide of the invention and the effect of candidate substances on those functions is an assay which measures spindle assembly and function. Typically, such assays are performed using Xenopus cell free systems, where two types of spindle assembly are possible. In the “half spindle” assembly pathway, a cytoplasmic extract of CSF arrested oocytes is mixed with sperm chromatin. The half spindles that form subsequently fuse together. A more physiological method is to induce CSF arrested extracts to enter interphase by addition of calcium, whereupon the DNA replicates and kinetochores form. Addition of fresh CSF arrested extract then induces mitosis with centrosome duplication and spindle formation (for discussion of these systems see Tournebize and Heald, 1996).

Again, generally, candidate substances known to bind to a polypeptide of the invention, or non-functional polypeptide variants of the invention, would be tested in this assay. Alternatively, a high throughput assay may be used to identify modulators of spindle formation and function and the resulting identified substances tested for affects binding of the polypeptide of the invention as described above.

Assays for DNA Replication

Another assay to investigate the function of polypeptide of the invention and the effect of candidate substances on those functions is as assay for replication of DNA. A number of cell free systems have been developed to assay DNA replication. These can be used to assay the ability of a substance to prevent or inhibit DNA replication, by conducting the assay in the presence of the substance. Suitable cell-free assay systems include, for example the SV-40 assay (Li and Kelly, 1984, Proc. Natl. Acad. Sci USA 81, 6973-6977; Waga and Stillman, 1994, Nature 369, 207-212.). A Drosophila cell free replication system, for example as described by Crevel and Cotteril (1991), EMBO J. 10, 4361-4369, may also be used. A preferred assay is a cell free assay derived from Xenopus egg low speed supernatant extracts described in Blow and Laskey (1986, Cell 47,577-587) and Sheehan et al. (1988, J. Cell Biol. 106, 1-12), which measures the incorporation of nucleotides into a substrate consisting of Xenopus sperm DNA or HeLa nuclei. The nucleotides may be radiolabelled and incorporation assayed by scintillation counting. Alternatively and preferably, bromo-deoxy-uridine (BrdU) is used as a nucleotide substitute and replication activity measured by density substitution. The latter assay is able to distinguish genuine replication initiation events from incorporation as a result of DNA repair. The human cell-free replication assay reported by Krude, et al (1997), Cell 88, 109-19 may also be used to assay the effects of substances on the polypeptides of the invention.

Other in vitro Assays

Other assays for identifying substances that bind to a polypeptide of the invention are also provided. For example, substances which affect chromosome condensation may be assayed using the in vitro cell free system derived from Xenopus eggs, as known in the art.

Substances which affect kinase activity or proteolysis activity are of interest. It is known, for example, that temporal control of ubiquitin-proteasome mediated protein degradation is critical for normal G1 and S phase progression (reviewed in Krek 1998, Curr Opin Genet Dev 8, 36-42). A number of E3 ubiquitin protein ligases, designated SCFs (Skp1-cullin-F-box protein ligase complexes), confer substrate specificity on ubiquitination reactions, while protein kinases phosphorylate substrates destined for destruction and convert them into preferred targets for ubiquitin modification catalyzed by SCFs. Furthermore, ubiquitin-mediated proteolysis due to the anaphase-promoting complex/cyclosome (APC/C) is essential for separation of sister chromatids during mitosis, and exit from mitosis (Listovsky et al., 2000, Exp Cell Res 255, 184-191).

Substances which inhibit or affect kinase activity may be identified by means of a kinase assay as known in the art, for example, by measuring incorporation of ³²P into a suitable peptide or other substrate in the presence of the candidate substance. Similarly, substances which inhibit or affect proteolytic activity may be assayed by detecting increased or decreased cleavage of suitable polypeptide substrates.

Assays for these and other protein or polypeptide activities are known to those skilled in the art, and may suitably be used to identify substances which bind to a polypeptide of the invention and affect its activity.

Whole Cell Assays

Candidate substances may also be tested on whole cells for their effect on cell cycle progression, including mitosis and/or meiosis. Preferably the candidate substances have been identified by the above-described in vitro methods. Alternatively, rapid throughput screens for substances capable of inhibiting cell division, typically mitosis, may be used as a preliminary screen and then used in the in vitro assay described above to confirm that the affect is on a particular polypeptide of the invention.

The candidate substance, i.e. the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. Preferably, the expression of the polypeptide is under the control of a regulatable promoter.

Typically, an assay to determine the effect of a candidate substance identified by the method of the invention on a particular stage of the cell division cycle comprises administering the candidate substance to a cell and determining whether the substance inhibits that stage of the cell division cycle. Techniques for measuring progress through the cell cycle in a cell population are well known in the art. The extent of progress through the cell cycle in treated cells is compared with the extent of progress through the cell cycle in an untreated control cell population to determine the degree of inhibition, if any. For example, an inhibitor of mitosis or meiosis may be assayed by measuring the proportion of cells in a population which are unable to undergo mitosis/meiosis and comparing this to the proportion of cells in an untreated population.

The concentration of candidate substances used will typically be such that the final concentration in the cells is similar to that described above for the in vitro assays.

A candidate substance is typically considered to be an inhibitor of a particular stage in the cell division cycle (for example, mitosis) if the proportion of cells undergoing that particular stage (i.e., mitosis) is reduced to below 50%, preferably below 40, 30, 20 or 10% of that observed in untreated control cell populations.

Therapeutic Uses

Many tumours are associated with defects in cell cycle progression, for example loss of normal cell cycle control. Tumour cells may therefore exhibit rapid and often aberrant mitosis. One therapeutic approach to treating cancer may therefore be to inhibit mitosis in rapidly dividing cells. Such an approach may also be used for therapy of any proliferative disease in general. Thus, since the polypeptides of the invention appear to be required for normal cell cycle progression, they represent targets for inhibition of their functions, particularly in tumour cells and other proliferative cells.

The term proliferative disorder is used herein in a broad sense to include any disorder that requires control of the cell cycle, or a disease whose pathogenesis involves abnormal cell proliferation. Examples of such diseases include neoplasias, such as sarcomas, carcinomas, lymphomas, blastomas, melanomas, myelomas, Wilms tumor, leukemias, adenocarcinomas, and the like, cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia.

One possible approach is to express anti-sense constructs directed against polynucleotides of the invention, preferably selectively in tumour cells, to inhibit gene function and prevent the tumour cell from progressing through the cell cycle. Anti-sense constructs may also be used to inhibit gene function to prevent cell cycle progression in a proliferative cell. Another approach is to use non-functional variants of polypeptides of the invention that compete with the endogenous gene product for cellular components of cell cycle machinery, resulting in inhibition of function. Alternatively, compounds identified by the assays described above as binding to a polypeptide of the invention may be administered to tumour or proliferative cells to prevent the function of that polypeptide. This may be performed, for example, by means of gene therapy or by direct administration of the compounds. Suitable antibodies of the invention may also be used as therapeutic agents.

Alternatively, double-stranded (ds) RNA is a powerful way of interfering with gene expression in a range of organisms that has recently been shown to be successful in mammals (Wianny and Zernicka-Goetz, 2000, Nat Cell Biol 2000, 2, 70-75). Double stranded RNA corresponding to the sequence of a polynucleotide according to the invention can be introduced into or expressed in oocytes and cells of a candidate organism to interfere with cell division cycle progression.

In addition, a number of the mutations described herein exhibit aberrant meiotic phenotypes. Aberrant meiosis is an important factor in infertility since mutations that affect only meiosis and not mitosis will lead to a viable organism but one that is unable to produce viable gametes and hence reproduce. Consequently, the elucidation of genes involved in meiosis is an important step in diagnosing and preventing/treating fertility problems. Thus the polypeptides of the invention identified in mutant Drosophila having meiotic defects (as is clearly indicated in the Examples) may be used in methods of identifying substances that affect meiosis. In addition, these polypeptides, and corresponding polynucleotides, may be used to study meiosis and identify possible mutations that are indicative of infertility. This will be of use in diagnosing infertility problems.

Administration

Substances identified or identifiable by the assay methods of the invention may preferably be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular or transdermal administration. Typically, each protein may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components (or antisense constructs) for use in inhibiting cell cycle progression, for example, inhibiting mitosis or meiosis, may be administered directly as a naked nucleic acid construct. They may further comprise flanking sequences homologous to the host cell genome. When the polynucleotides/vectors are administered as a naked nucleic acid, the amount of nucleic acid administered may typically be in the range of from 1 μg to 10 mg, preferably from 100 μg to 1 mg. It is particularly preferred to use polynucleotides/vectors that target specifically tumour or proliferative cells, for example by virtue of suitable regulatory constructs or by the use of targeted viral vectors.

Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™ ). Typically, nucleic acid constructs are mixed with the transfection agent to produce a composition.

Preferably the polynucleotide, polypeptide, compound or vector described here may be conjugated, joined, linked, fused, or otherwise associated with a membrane translocation sequence.

Preferably, the polynucleotide, polypeptide, compound or vector, etc described here may be delivered into cells by being conjugated with, joined to, linked to, fused to, or otherwise associated with a protein capable of crossing the plasma membrane and/or the nuclear membrane (i.e., a membrane translocation sequence). Preferably, the substance of interest is fused or conjugated to a domain or sequence from such a protein responsible for the translocational activity. Translocation domains and sequences for example include domains and sequences from the HIV-1-trans-activating protein (Tat), Drosophila Antennapedia homeodomain protein and the herpes simplex-1 virus VP22 protein. In a highly preferred embodiment, the substance of interest is conjugated with penetratin protein or a fragment of this. Penetratin comprises the sequence RQIKIWFQNRRMKWKK and is described in Derossi, et al., (1994), J. Biol. Chem. 269, 10444-50; use of penetratin-drug conjugates for intracellular delivery is described in WO/00/01417. Truncated and modified forms of penetratin may also be used, as described in WO/00/29427.

Preferably the polynucleotide, polypeptide, compound or vector according to the invention is combined with a pharmaceutically acceptable carrier or diluent to produce a pharmaceutical composition. Suitable carriers and diluents include isotonic saline solutions, for example phosphate-buffered saline. The composition may be formulated for parenteral, intramuscular, intravenous, subcutaneous, intraocular or transdermal administration.

The routes of administration and dosages described are intended only as a guide since a skilled practitioner will be able to determine readily the optimum route of administration and dosage for any particular patient and condition.

The invention will now be further described by way of Examples, which are meant to serve to assist one of ordinary skill in the art in carrying out the invention and are not intended in any way to limit the scope of the invention.

### EXAMPLES

Generation and Identification of Lethal, Semi-Lethal and Sterile Third Chromosome Mutants Having Defects in Mitosis and/or Meiosis, and Second Chromosome Mutants Having Defects in Imaginal Disc Development by P-Element Insertion Mutagenesis

P-element Mutagenesis

Transposable elements are widely used for mutagenesis in Drosophila melanogaster as they couple the advantages of providing effective genetic lesions with ease of detecting disrupted genes for the purpose of molecular cloning. To achieve near saturation of the genome with mutations resulting from mobilisation of the P-lacW transposon (a P-element marked with a mini-white gene, bearing the E. coli lacZ gene as an enhancer trap, and an E. coli replicon and ampicillin resistance gene to facilitate ‘plasmid rescue’ of sequences at the site of the P-insertion), Drosophila females that are homozygous for P-lacW (inserted on the X chromosome) are crossed with males carrying the transposase source P(Δ2-3) (Deak et al., 1997). Random transpositions of the mutator element are then ‘captured’ in lines lacking transposase activity. Stable, or balanced, stocks bearing single lethal P-lacW insertions are made.

More than 41,000 lines are derived, of which approximately one-half are on the third chromosome. Originally some 3100 lethal or strong semi-lethal lines (in homozygous conditions) are identified. During preliminary characterisation unstable lines and clusters of the same mutation event are eliminated leaving 2460 lines to be characterised.

Screening for Mitotic and Meiotic Defects

About half of the mutants in the collection are embryonic lethals. We have carried out cytological screens of the 1155 lines that comprise late larval lethals, pupal lethals, pharate and adult semi-lethals for defective mitosis in the developing larval CNS. This has identified 69 mutations falling into 43 complementation groups that affect all stages of the mitotic cycle. The cytological screens involve examining orcein-stained squashed preparations of the larval CNS to detect abnormal mitotic cells. In lines where defects are identified, the larval CNS is subjected to immunostaining to identify centromeres, spindle microtubules and DNA for further examination. This leads to clarification of the mitotic defect.

As a set of common functions are essential to both mitosis and meiosis, we then identify mutations resulting in sterility and failed progression through male meiosis. This involves examining squashed preparations larval, pupal or adult testes by phase contrast microscopy. We examine “onion stage” spermatids in the 519 pupal and pharate lethal lines and 463 adult “semi-lethal” and viable lines for variations in size and number of nuclei which provides an indication of whether there have been defects in either chromosome segregation or cytokinesis, respectively. A total of 54 lines of the 519 pupal and pharate lethal lines and 22 of the adult lines show such defects. However, another 67 lines show male sterility without having onion-stage defects. 12 lines showing onion stage defects have been scored as having mitotic defects in the independent cytological screen of squashed preparations of the larval CNS. Twelve further lines with onion stage defects show female sterility and of these, 10 show maternal effect mitotic defects in syncytial embryos. Thus greater than one half of the meiotic mutants scored appear to represent cell division functions specific to male meiosis or have targeted male germ-line specific enhancer elements, thus revealing their meiotic function but in this test not their mitotic function.

Further characterisation of testis preparations of each line by phase-contrast microscopy with and without staining with Hoechst to reveal DNA defined 6 broad categories of meiotic mutants:

8 mutants from the collection show defects in meiotic entry or at early stages in the first meiotic division (MF1-8).

18 mutants (15 complementation groups) show abnormal meiotic spindles (AB1-16). Mutants in this group almost invariably show an associated weak defect in cytokinesis, and 7 show a strong defect in spermatid differentiation. 3 of these mutants also show mitotic defects in larval brains or in embryos derived from homozygous mutant mothers.

18 mutants (16 complementation groups) also show abnormal meiotic spindles that are strongly multipolar (MUL1-15). Three of these also show maternal effect mitotic abnormalities of multipolar spindles in syncytial embryos.

4 mutants (3 complementation groups) show strong defects at all stages of spermatogenesis from the pre-meiotic stages to spermatid elongation stages (PL1-3). In this respect they resemble the polo¹ mutation.

4 mutants show segregation defects as indicated by spermatid nuclei of heterogeneous sizes (SEG1-4). The spindles appear normal but all have what are either chromosome bridges or lagging chromosomes. One of these also shows a maternal effect.

9 mutants (7 complementation groups) show predominant cytokinesis defects. Two complementation groups also have cytokinesis defects in mitotic cells in the larval brain.

In the Examples below, the designations MF, AB, MUL, PL, SEG or CK are included in the category description where available. Further phenotype information for each mutant described in the results section is provided in the “Phenotype” field. There is considerable overlap between these categories, and it will be of much interest to distinguish between mutants in which the primary defect results in secondary consequences, and mutants that affect more than one aspect of spermatogenesis, as for example appears to be the case with polo mutants (Sunkel and Glover, 1988; Carmena et al, 1998).

In the Examples, lines exhibiting mitotic and meiotic phenotypes are categorised generally into four categories:

Category 1: Failure to complete cytokinesis

Category 2: Failure to enter M-phase

Category 3: Metaphase arrest

Category 4: Anaphase defect

Category 5: Small Imaginal Discs (Block to Proliferation; see below)

Category 1 phenotypes are exhibited by mutations in Examples 1 to 14; while Category 2 phenotypes are exhibited by mutations in Examples 15 to 19. Category 3 phenotypes are exhibited by mutations in Examples 20 to 30, Category 4 phenotypes are exhibited by mutations in Examples 31 to 53. Mutations in Examples 54 to 74 exhibit a Category 5 phenotype.

Generation and Identification of Second Chromosome Mutants Having Small or No Imaginal Discs.

In the case of the second chromosome the flies used were from a second chromosome P-element collection established in Szeged, Hungary (Torok et al., 1993). The process of P-element insertion mutagenesis is essentially as described above. 15475 insertions were recovered, of which 2711 were lethal or semi-lethal. After elimination of clusters of identical mutants, 2399 lines representing 1748 independent lethal insertions were recovered. Lines were chosen from the second chromosome collection on the basis of having small or no imaginal discs, to indicate a disruption in cell cycle progression that leads to underdevelopment of the discs. All the second chromosome mutants referred to in the results section are noted under the “Phenotype” field as “second chromosome, small imaginal discs” and comprise Category 5.

Cytological Mapping of the P-Element Insertion Sites

The site of insertion of the P-element in each mutant line was determined by in situ hybridisation of P-element DNA to salivary gland polytene chromosomes as described in Saunders et al., 1989. Wandering third stage larvae were dissected and fixed as described and incubated with biotin-labeled DNA made from the P-lacW plasmid. After signal detection chromosomes were stained with Giemsa and examined by microscopy and signals indicating the presence of P elements were assigned to polytene chromosome bands referring to the Bridges map (Lefevre, 1976). In the majority of cases a single P element was detected, only 10% of lines having multiple (two or three) insertions. The site of insertion is given as the “Map Position” field in the results section (for example 77B)

Plasmid Rescue of P-Elements from Mutant Drosophila Lines

Genomic DNA was isolated from adult flies by the method of Jowett et al., 1986, and plasmid rescue from the genomic DNA was performed according to Pirrotta et al., 1986. This allows the recovery of genomic DNA adjacent to the P-element which facilitates the identification of the site of P-element insertion and of genes which may be disrupted by the insertion. Essentially, genomic DNA derived from about 200 flies was digested with a restriction enzyme known to have a site within the P-element (EcoR1 or SacII for cloning sequences to the left of the element, or XbaI, BglII, PstI or BamHI for sequences to the right of the element). The digested DNA was ligated overnight, and plasmids recovered by electroporation of the ligated DNA into E. coli XL1-blue competent cells. Appropriate primers from within the P-lacW sequence were used to determine the sequence of the genomic DNA flanking the element (on average, 400 bp of sequence were obtained). The rescue sequences are provided in the results section under the heading “Rescue sequence”. Where more than one sequence was recovered, the orientation of each sequence is also given.

Sequence Analysis of P Element Insertion Lines

Sequences flanking the insertion site of the P-element were derived by P element rescue as described above. In some cases sequence was obtained from only one side of the insertion, while in other cases sequences were obtained from both sides of the insertion.

As a first step, each P element rescue sequence was used to search a total database of Drosophila melanogaster sequences (database of the Berkley Drosophila Genome project) using the BLASTN program (which compares a nucleic acid sequence with a nucleic acid database, (Altschul and Lipman 1990)) with default parameters.

The search may identify a number of different types of match including Drosophila ESTs, known Drosophila genes and cloned genomic regions.

The ability to identify genes already known to be essential for cell cycle progression using this approach was confirmed, in this example, by the rescue sequence obtained from line 1324/8 which mapped to the 77B locus which was used to search the database. A BLASTN search identified a number of matching Drosophila ESTs, a match with the known cell cycle regulatory gene polo and a cloned genomic region designated CSC: AC018188. These matches are recorded in the results sections under the field headings “Drosophila ESTs”, “Drosophila gene hit” and “Genomic hit, Accession No.”, respectively. Any entries under “Drosophila gene hit” are further annotated with “(BLASTN with Rescue sequence)” to show that the match was obtained using the rescue sequence rather than a Drosophila EST or genomic clone ORF (see below). Accession numbers of ESTs, genes and genomic clones are provided where known. Genomic clones designations often include the Genbank designation as part of a longer designation. However the Genbank designation is always the code beginning with “AC” and followed by six digits.

Where an EST was identified, this was subsequently used to search using the BLASTX program (default parameters) against databases of sequences from Drosophila and Homo sapiens (databases of the National centre for Biotechnology Information (NCBI), National Library of Medicine, National Institue of Health, USA). In the case of line 1104/16, the search identified a known human gene, phosphatidylinositol transfer protein (accession no. P48739) implying a novel function for this protein in cytokinesis. Human Homologues identified as a result of a BLASTX search using a Drosophila EST are shown in the results section under the heading “Human homologues” and annotated with “(BLASTX with EST)”. Drosophila genes identified as a result of a BLASTX search using a Drosophila EST are shown in the results section under the heading “Drosophila gene hit” and annotated with “(BLASTX with EST)”.

Where no Drosophila gene was identified using the initial BLASTN search but a matching genomic clone was found (a Bac or P1 clone often in excess of 100 kilobases), a 20 kilobase segment of this genomic sequence (10 kilobases either side flanking the site of the P-element insertion) was subjected to a number of analyses.

If the rescue sequence matched sequences that lie within a known gene present within the genomic clone then these are presented under the heading “Drosophila gene hit (BLASTN with Rescue sequence”. The known gene sequence was then used in a BLASTX search of a human database (NCBI—see above) to identify any human homologues. These are shown in the “Human homologue” field and annotated with “(BLASTX with Drosophila gene)”.

If the rescue sequence does not match any sequences that lie with a known gene within the genomic clone then the occurrence of ORFs within the 20 kilobase genomic segment was predicted using the Genscan programme (Burge and Karlin, 1997). Where the P-element was observed to be inserted into the coding region or within the 5′ untranslated region (which we defined as within 2 kilobases of the predicted start of the coding region) we assume the P element to be capable of disrupting the expression of the predicted gene. Each predicted open reading frame (or predicted coding sequence) was then used to search Drosophila and human databases using the TBLASTN program (compares a protein query sequence against a nucleotide sequence database dynamically translated in all reading frames) and/or the TBLASTX program (compares a nucleotide query sequence dynamically translated in all reading frames against a nucleotide sequence database dynamically translated in all reading frames) to determine whether the predicted open reading frame corresponded to a known gene. Typically, TBLASTX is only used when no matches are found using TBLASTN.

Where the TBLASTN search found a known Drosophila gene, then this is indicated in the results in the “Drosophila gene hit” field, annotated with “(TBLASTN with predicted ORF)”. The Drosophila gene sequence was then typically used to search a human database (NCBI—see above) to identify any human homologues using BLASTX. These are shown in the “Human homologue” field and annotated with “(BLASTX with Drosophila gene)”.

Where the TBLASTN and/or TBLASTX search found a known human gene, then this is indicated in the results in the “Human homologue” field, annotated with “(TBLASTN (or TBLASTX) with predicted ORF)”.

If the TBLASTN and/or TBLASTX search found no Drosophila or human genes, then it was assumed that the original ORF corresponds to a novel gene. If the TBLASTN search found no Drosophila genes but identified a human homologue, then it was assumed that the original ORF corresponds to a novel Drosophila homologue of a known human gene.

Additional Sequence Analysis Using the Annotated D. melanogaster Sequence (GadFly).

Rescue sequences were also used to search the fully annotated version of the Drosophila genome (GadFly; Adams, et al., 2000, Science 287, 2185-2195), using GlyBLAST at the Berkeley Drosophila Genome Projects web site to identify the genome segment (usually approximately 200-250 kb) containing the P-element insertion site. The graphic representation of the genomic fragment available at GadFly allows the identification of all real and theoretical genes which flank the site of insertion. Candidate genes where the P-element is either inserted within the gene or close to the 5′ end of the gene were identified. In GadFly, the Drosophila genes are given the designation CG (Complete gene) and usually details of human homologues are also given. In most cases, this data confirms the data derived from the sequence analysis procedure described above, and in some cases new data is obtained. Where available both sets of data are included in the individual Examples described below. To identify further candidate human homologues, BLASTP (amino acid query sequence against amino acid database) searches with Drosophila sequences are used against the human genome project database and also the Ensembl dataset. The Ensembl dataset comprises GeneWise gene predictions using a protein template where possible or Genscan followed by BLAST confirmation via protein, cDNA or EST hits. These are matched using WUBLASTP with default parameters (Altschul et al., 1990, J Mol Biol 215, 403-10). The results are filtered to contain only potential homologues. Only matches with the identity of more than 50% and length of more than 50 amino acids are included.

Confirmation of Cell Cycle Involvement of Candidate Genes Using Double Stranded RNA Interference (RNAi)

P-elements usually insert into the region 5′ to a Drosophila gene. This means that there is sometimes more than one candidate gene affected, as the P-element can insert into the 5′ regions of two diverging genes (one on each DNA strand). In order to confirm which of the candidate genes is responsible for the cell cycle phenotype observed in the fly line, we use the technique of double stranded RN interference to specifically knock out gene expression in Drosophila cells in tissue culture (Clemens, et al., 2000, Proc. Natl. Acad. Sci. USA, 6499-6503). The overall strategy is to prepare double stranded RNA (dsRNA) specific to each gene of interest and to transfect this into Schneider's Drosophila line 2 to inhibit the expression of the particular gene. The dsRNA is prepared from a double stranded, gene specific PCR product with a T7 RNA polymerase binding site at each end. The PCR primers consist of 25-30 bases of gene specific sequence fused to a T7 polymerase binding site (TAATACGACTCACTATAGGGACA), and are designed to amplify a DNA fragment of around 500 bp. Although this is the optimal size, the sequences in fact range from 450 bp to 650 bp. Where possible, PCR amplification is performed using genomic DNA purified from Schneider's Drosophila line 2 as a template. This is only feasible where the gene has an exon of 450 bp or more. In instances where the gene possesses only short exons of less than 450 bp, primers are designed in different exons and PCR amplification is performed using cDNA derived from Schneider's Drosophila line 2 as a template.

A sample of PCR product is analysed by horizontal gel electrophoresis and the DNA purified using a Qiagen QiaQuick PCR purification kit. 1 μg of DNA is used as the template in the preparation of gene specific single stranded RNA using the Ambion T7 Megascript kit. Single stranded RNA is produced from both strands of the template and is purified and immediately annealed by heating to 90 degrees C. for 15 mins followed by gradual cooling to room temperature overnight. A sample of the dsRNA is analysed by horizontal gel electrophoresis.

3 μg of dsRNA is transfected into Schneider's Drosophila line 2 using the transfection agent, Transfect (Gibco) and the cells incubated for 72 hours prior to fixation. The DNA content of the cells is analysed by staining with propidium iodide and standard FACS analysis for DNA content. The cells in G1 and G2/S phases of the cell cycle are visualised as two separate population peaks in normal cycling S2 cells. In each experiment, Red Fluorescent Protein dsRNA is used as a negative control. In some cases the phenotype is confirmed by fixing cells on poly-lysine covered slides which are then stained for DNA using DAPI and for tubulin using an anti-tubulin antibody YL1/2 and appropriate fluorescent secondary antibody to visualise aberrant mitoses.

It should be noted that RNAi could not confirm phenotype in all cases. This is to be expected as the method relies on the ability of dsRNA to prevent new protein expression. Consequently, it is necessary that S2 cells express the specific cDNA of the gene in question, and also that the protein is turned over rapidly. It would therefore probably be difficult to sufficiently reduce levels of very stable proteins using this approach.

Alternatively, or in addition, the technique of interference using short interfering RNAs (siRNA), as described in Elbashir et al, Nature 2001 May 24;411(6836):494-8, may be used to confirm cell cycle involvement of any of the genes in question. The siRNA technique may be conducted in a similar fashion to an RNAi procedure, for example as described above, except that typically shorter fragments of double stranded RNA are employed, for example, shorter than 500 bases, typically 10 to 400 bases, preferably between 10 to 100 bases, preferably between 10 to 50 bases, preferably about 20 bases or so, for example 21 bases.

A specific siRNA technique for knockdown of gene expression is described in detail at Example 6.

[image]

[image]

[image]

[image]

[image]

[image]

[image]

[image]

[image]

[image]

[image]

[image]

[image]

[image]

[image]

[image]

[image]

[image]

### Example 6A

Identification of Pebble

Pebble as a candidate gene is detected in a screen of a P-element insertion library covering the 3^{rd} chromosome of Drosophila melanogaster (Deak et al 1997) as mutant phenotype in fly line 293/9, as described above.

Mitotic defects are observed in brain squashes: a very high occurrence of polyploidy suggesting a cytokinesis defect.

Rescue and sequencing of genomic DNA flanking the P-element insertion site indicates that the P-element is inserted into the 5′ region of gene CG8114, the pebble (pbl) rho1 GTPase exchange factor.

### Example 6B

Validation of Pebble Function by RNA Interference (RNAi) Knockdown in Drosophila Cultured Cells

To confirm the mitotic role of the target protein, knockdown of pebble expression is performed in cultured Drosophila Dmel-2 cells using a double stranded RNA (dsRNA) from within the pebble CDS corresponding to the following pebble CDS sequence 1814 to 2408 (ttgcacg . . . to . . . aagcacgg).

| | |
|---|---|
| 1814 | ttgcacg ctaactggcg ggaggattgt cttattggag acattatcat |
| 1861 | tcaacatcgc gacgagttga taaaggcgta tccaccatac gtcaacttct tcgagcagat |
| 1921 | gaaggagcaa ctgcagtact gcgatcggga atatccgcgt ttccacgcct ttctgaagat |
| 1981 | taatcaaacg aaaccggagt gcggacgcca aggtctacaa gatctgatga tccgtccggt |
| 2041 | tcagcgattg cccagtatca gtctgctgct aaatgatata ttgaaacaca cgactagtgg |
| 2101 | caatgcggac cacggacgtt tggaggaggc cctgaaggcc atcaagcagg tgacactgca |
| 2161 | catcaacgag gataaaaggc ggaccgaatc gcgcatggcc atcttcgata tattcaacga |
| 2221 | tatcgaagga tgtccggcgc atttagtgag ttccaaccgc agctttattt taaagtgtga |
| 2281 | ggtaaacgag ctctccgatt cgctgagcgg tcgtggtgat agcctggtcc tgtacctgtt |
| 2341 | ctccgattcc attgagctgt gcaagcggcg ttctaaagga ttcaacactg caaaatcgcc |
| 2401 | aagcacgg |

dsRNA is prepared by annealing complimentary RNAs made by in vitro transcription. Cells are transfected with double stranded RNA in the presence of ‘Transfast’ transfection reagent. A control transfection of a non-endogenous RNA corresponding to RFP (red fluorescent protein) is carried out in parallel.

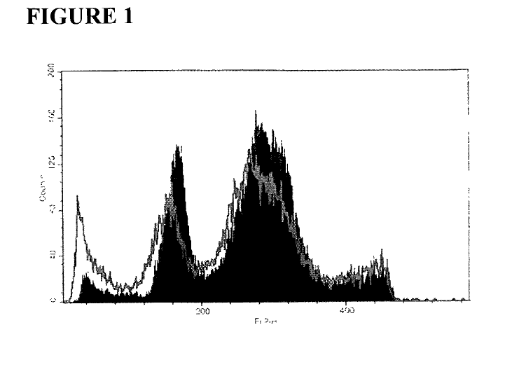

Cells are fixed after 3 days and stained with propidium iodide for subsequent FACS analysis. Results are shown in FIG. 1. Cells show an overall reduction in G1 and G2/M and an increase in sub-G1 cells indicating cell death or exit from cycle, but no significant change in G1/G2M compartments.

### Example 6B

Ect2 is a Human Homologue of Drosophila Pebble

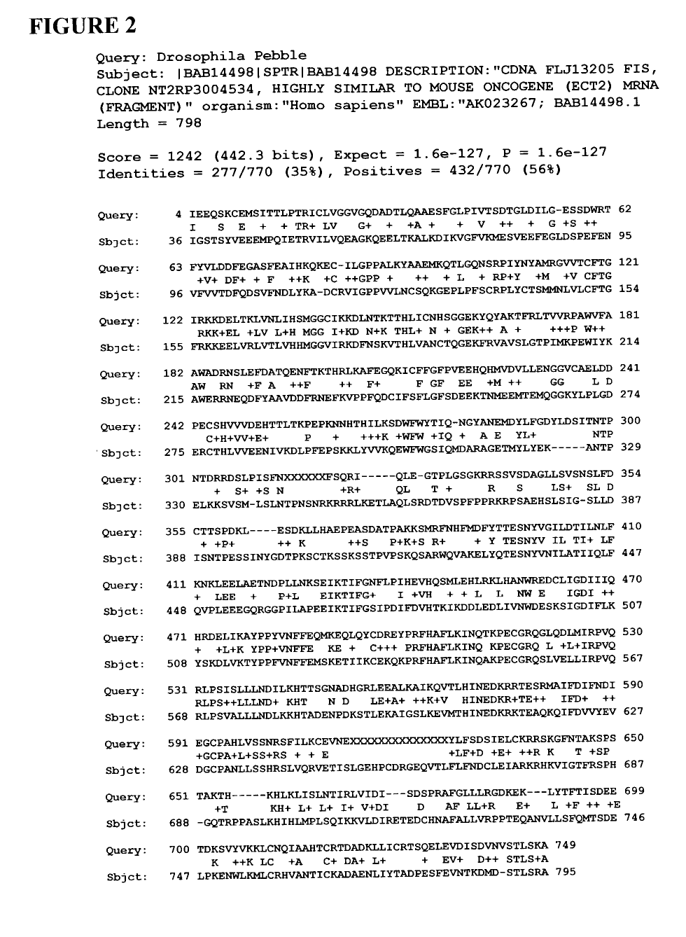

BLASTX with pebble reveals 35% sequence identity with Ect2 (AK023267), indicating that Ect2 is a homologue of Drosophila pebble. The BLASTX results are shown in FIG. 2.

The sequence of human Ect2 is shown below:

agagtgctgatttagaagaatacaaatcatggctgaaaatagtgtattaacatccactactgggaggactag
cttggcagactcttccatttttgattctaaagttactgagatttccaaggaaaacttacttattggatctac
ttcatatgtagaagaagagatgcctcagattgaaacaagagtgatattggttcaagaagctggaaaacaaga
agaacttacaaaagccttaaaggacattaaagtgggctttgtaaagatggagtcagtggaagaatttgaagg
tttggattctccggaatttgaaaatgtatttgtagtcacggactttcaggattctgtctttaatgacctcta
caaggctgattgtagagttattggaccaccagttgtattaaattgttcacaaaaaggagagcctttgccatt
ttcatgtcgcccgttgtattgtacaagtatgatgaatctagtactatgctttactggatttaggaaaaaaga
agaactagtcaggttggtgacattggtccatcacatgggtggagttattcgaaaagactttaattcaaaagt
tacacatttggtggcaaattgtacacaaggagaaaaattcagggttgctgtgagtctaggtactccaattat
gaagccagaatggatttataaagcttgggaaaggcggaatgaacaggatttctatgcagcagttgatgactt
tagaaatgaatttaaagttcctccatttcaagattgtatttttagtttcctgggattttcagatgaagagaa
aaccaatatggaagaaatgactgaaatgcaaggaggtaaatatttaccgcttggagatgaaagatgcactca
ccttgtagttgaagagaatatagtaaaagatcttccctttgaaccttcaaagaaactttatgttgtcaagca
agagtggttctggggaagcattcaaatggatgcccgagctggagaaactatgtatttatatgaaaaggcaaa
tactcctgagctcaagaaatcagtgtcaatgctttctctaaatacccctaacagcaatcgcaaacgacgtcg
tttaaaagaaacacttgctcagctttcaagagatacagacgtgtcaccatttccaccccgtaagcgcccatc
agctgagcattccctttccatagggtcactcctagatatctccaacacaccagagtctagcattaactatgg
agacaccccaaagtcttgtactaagtcttctaaaagctccactccagttccttcaaagcagtcagcaaggtg
gcaagttgcaaaagagctttatcaaactgaaagtaattatgttaatatattggcaacaattattcagttatt
tcaagtaccattggaagaggaaggacaacgtggtggacctatccttgcaccagaggagattaagactatttt
tggtagcatcccagatatctttgatgtacacactaagataaaggatgatcttgaagaccttatagttaattg
ggatgagagcaaaagcattggtgacatttttctgaaatattcaaaagatttggtaaaaacctaccctccctt
tgtaaacttctttgaaatgagcaaggaaacaattattaaatgtgaaaaacagaaaccaagatttcatgcttt
tctcaagataaaccaagcaaaaccagaatgtggacggcagagccttgttgaacttcttatccgaccagtaca
gaggttacccagtgttgcattacttttaaatgatcttaagaagcatacagctgatgaaaatccagacaaaag
cactttagaaaaagctattggatcactgaaggaagtaatgacgcatattaatgaggataagagaaaaacaga
agctcaaaagcaaatttttgatgttgtttatgaagtagatggatgcccagctaatcttttatcttctcaccg
aagcttagtacagcgggttgaaacaatttctctaggtgagcacccctgtgacagaggagaacaagtaactct
cttcctcttcaatgattgcctagagatagcaagaaaacggcacaaggttattggcacttttaggagtcctca
tggccaaacccgacccccagcttctcttaagcatattcacctaatgcctctttctcagattaagaaggtatt
ggacataagagagacagaagattgccataatgcttttgccttgcttgtgaggccaccaacagagcaggcaaa
tgtgctactcagtttccagatgacatcagatgaacttccaaaagaaaactggctaaagatgctgtgtcgaca
tgtagctaacaccatttgtaaagcagatgctgagaatcttatttatactgctgatccagaatcctttgaagt
aaatacaaaagatatggacagtacattgagtagagcatcaagagcaat

A deduced amino acid sequence is shown below:

MAENSVLTSTTGRTSLADSSIFDSKVTEISKENLLIGSTSYVEEEMPQIETRVILVQEAGKQE
ELTKALKDIKVGFVKMESVEEFEGLDSPEFENVFVVTDFQDSVFNDLYKADCRVIGPPVVLNCSQK
GEPLPFSCRPLYCTSMMNLVLCFTGFRKKEELVRLVTLVHHMGGVIRKDFNSKVTHLVANCTQGE
KFRVAVSLGTPIMKPEWIYKAWERRNEQDFYAAVDDFRNEFKVPPFQDCIFSFLGFSDEEKTNMEE
MTEMQGGKYLPLGDERCTHLVVEENIVKDLPFEPSKKLYVVKQEWFWGSIQMDARAGETMYLYE
KANTPELKKSVSMLSLNTPNSNRKRRRLKETLAQLSRDTDVSPFPPRKRPSAEHSLSIGSLLDISNTP
ESSINYGDTPKSCTKSSKSSTPVPSKQSARWQVAKELYQTESNYVNILATIIQLFQVPLEEEGQRGGPI
LAPEEIKTIFGSIPDIFDVHTKIKDDLEDLIVNWDESKSIGDIFLKYSKDLVKTYPPFVNFFEMSKETIIK
CEKQKPRFHAFLKINQAKPECGRQSLVELLIRPVQRLPSVALLLNDLKKHTADENPDKSTLEKAIGS
LKEVMTHINEDKRKTEAQKQIFDVVYEVDGCPANLLSSHRSLVQRVETISLGEHPCDRGEQVTLFLF
NDCLEIARKRHKVIGTFRSPHGQTRPPASLKHIHLMPLSQIKKVLDIRETEDCHNAFALLVRPPTEQA
NVLLSFQMTSDELPKENWLKMLCRHVANTICKADAENLIYTADPESFEVNTKDMDSTLSRASRA

### Example 6C

Validation of the Mitotic Role of the Human Homologue by siRNA Knockdown of Ect2 Expression in Human Cultured Cells

Generation of siRNA Ect2 Knockdowns

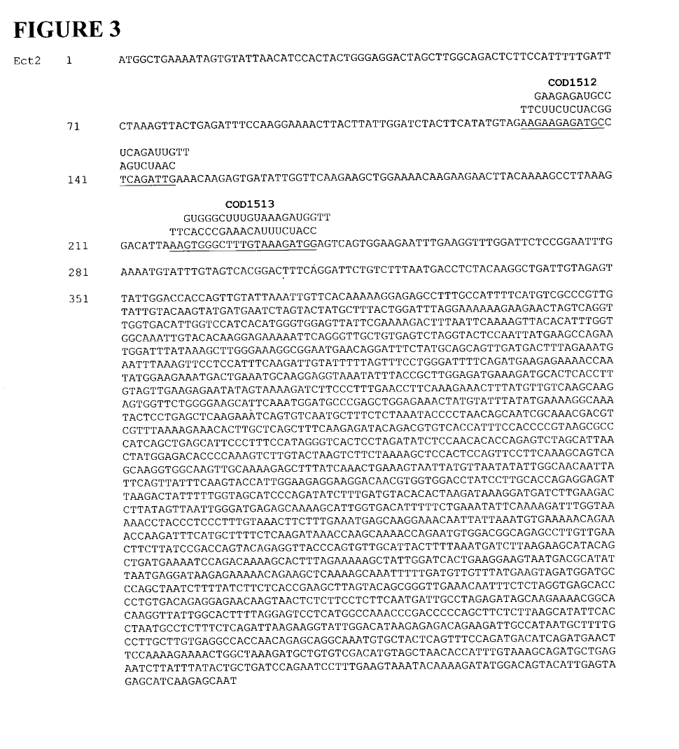

Knockdown of Ect2 gene expression is achieved by siRNA (short interfering RNA, Elbashir et al, Nature 2001 May 24;411(6836):494-8). We used synthetic double stranded RNAs (COD1512 and COD1513, as shown in FIG. 3) corresponding to two different regions in the 5′ region of the Ect2 mRNA. siRNAs are obtained from Dharmacon (our supplier).

The siRNA sequences of COD1512 and COD1513 are:

| | |
|---|---|
| COD1512: | ^{5′} GAAGAGAUGCCUCAGAUUGTT^{3′} |
| | ^{3′}TTCUUCUCUACGGAGUCUAAC ^{5′} |
| COD1513: | ^{5′} GUGGGCUUUGUAAAGAUGGTT^{3′} |
| | ^{3′}TTCACCCGAAACAUUUCUACC ^{5′} |

Protocol for siRNA Transfection for Western Blot Analysis

Cells are seeded in 6-well tissue culture dishes at 4×10⁵ cells/well in 2 ml Dulbecco's Modified Eagle's Medium (DMEM) (Sigma)+10% Foetal Bovine Serum (FBS) (Perbio), and incubated overnight (37° C./5% CO₂).

For each well, 12 μl of 20 μM siRNA duplex (Dharmacon, Inc) (in RNAse-free H₂O) is mixed with 200 μl of Optimem (Invitrogen). In a separate tube 8 μl of oligofectamine reagent (Invitrogen) was mixed with 52 μl of Optimem, and incubated at room temperature for 7-10 mins. The oligofectamine/Optimem mix is then added dropwise to the siRNA/Optimem mix, and this is then mixed gently, before being incubated for 15-20 mins at room temperature. During this incubation the cells are washed once with DMEM (no FBS or antibiotics added). 600 μl of DMEM (no FBS or antibiotics) is then added to each well.

Following the 15-20 min incubation, 128 μl of Optimem is added to the siRNA/oligofectamine/optimem mix, and this was added to the cells (in 600 μl DMEM). The transfection mix is added at the edge of each well to assist dilution before contact is made with the cells. Cells are then incubated with the transfection mix for 4 h (37° C./5%CO₂). Subsequently 1 ml DMEM+20% FBS is added to each well. Cells are then incubated at 37° C./5% CO₂ for the required period (24-72 h), before harvesting for Western blot analysis. Duplicate transfections are performed for each siRNA duplex, which are subsequently pooled, prior to production of cell lysates.

At the appropriate time point media is removed and replaced with 1 ml of cold Dulbecco's Phosphate Buffered Saline (PBS) (Sigma). Cells are then harvested by cell scraping. The cells from duplicate transfections are pooled, and then pelleted by centrifugation. The cell pellet is resuspended in 80 82 l of 1×Cell Lysis Buffer (Promega) with vortexing. Cells are lysed by multiple freeze thaw cycles.

The protein content of the supernatant is determined and equal amounts of protein are assayed by Western blotting.

Protocol for siRNA Transfection for Flow Cytometry Analysis

The transfection method for samples for flow cytometry analysis is identical to that for Western blot, except that cells are initially seeded at 4×10⁵ cells/well, and transfections are performed in triplicate, and pooled prior to analysis.

Cells are harvested by trypsinisation, washed in PBS, fixed in ice-cold 70% EtOH and stained with Propidium Iodide before loading into the FACs machine.

Analysis of siRNA Ect2 Knockdowns in 293 Cells by Western Blot

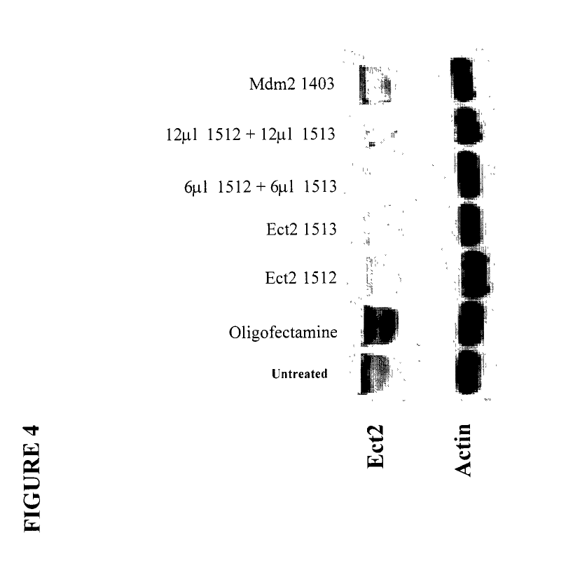

293 is a human embryonic kidney 293 cell line that has been transformed with sheared Ad5 DNA, and is a commonly used cultured human cell line. 293 cells are incubated with siRNAs for 72 hours and knockdown of protein levels checked by Western blot, as shown in FIG. 4.

Expression levels of Ect2 and actin (control) are detected using relevant antibodies: anti-Ect2 (Supplier: Santa Cruz Biotechnology, Inc; Antibody name: Ect2 (C20); Product No.: sc-1005) and anti-actin (Supplier: Sigma; Antibody name: Monoclonal Anti-β-Actin; clone AC-15; Product No.: A 5441).

Analysis of siRNA Ect2 Knockdowns in U2OS Cells by FACs

siRNA Ect2 knockdowns are conducted in U2OS cells (human osteosarcoma) and analysed by FACs.

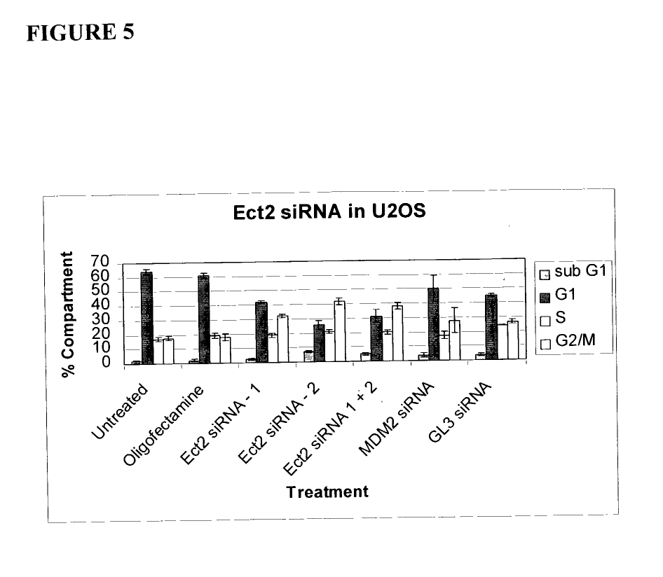

As shown in FIG. 5 (FACs), major changes in expression in difference cell cycle compartments (G1, S, G2 and M) are seen with siRNA-1 (COD1512), Ect2 siRNA-2 (COD1513) and treatment with both Ect siRNA 1+2. An accumulation of cells in the G2/M compartment of the cell cycle is observed with a concomitant reduction in the G1 compartment population.

Subsequent microscopic analysis is performed in order to phenotype the Ect siRNA induced defect and check for the presence of large multinucleate cells which may, due to their size and ploidy, be gated out of the FACS analysis.

Analysis of siRNA Ect2 Knockdowns in U2OS Cells by Microscopy

Phenotype analysis by microscopy is conducted on U2OS cells (human osteosarcoma).

Cells are incubated with siRNA for 48 hours before fixation and co-staining with Dapi to reveal DNA (blue) and antibodies to reveal microtubules (green). Antibodies used are mouse anti-gamma-tubulin (GTU88) secondary antibody Alexagreen488-goat anti-mouseIgG from Sigma to reveal centrosomes and rat anti-alphatubulin (YL12) Serotec and goat antirat IgG-TRITC from Jackson Immunoresearch

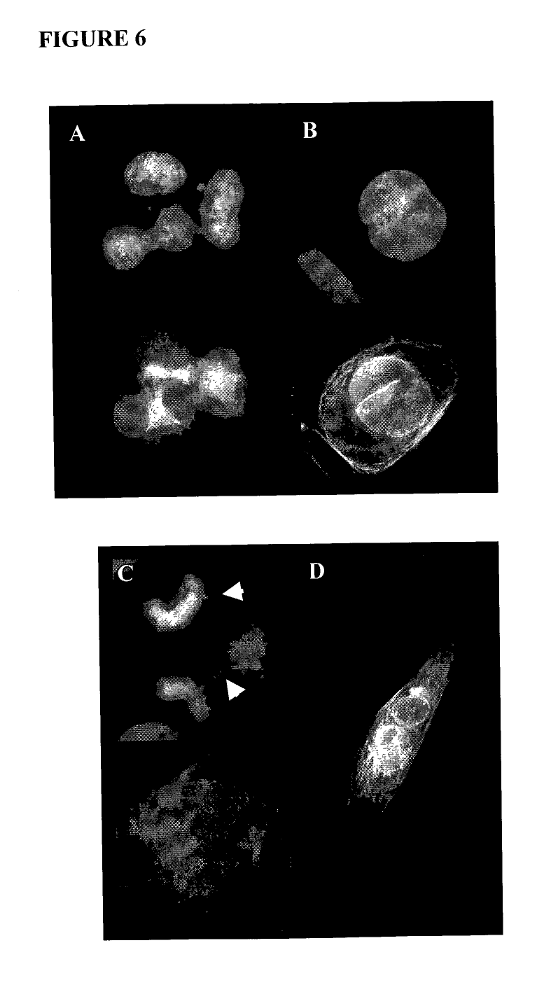

Results from duplicate experiments in U2OS cells are shown in FIG. 6.

The phenotypes displayed by siRNA Ect2 knockdowns of human cells include significant (8 fold) increase in multinucleated cells (observed mainly as large & possibly fused nuclei), as well as the presence of numerous binuclear cells. There is a significant decrease in confluency (confluency only 25% of control cells after 48 hr), and the presence of 23% mitotic defects. Of these the most dominant type are 35% with lagging chromatin.

Analysis of siRNA Ect2 Knockdowns in HeLa Cells by Microscopy

Similar phenotype analysis is also conducted using HeLa cells.

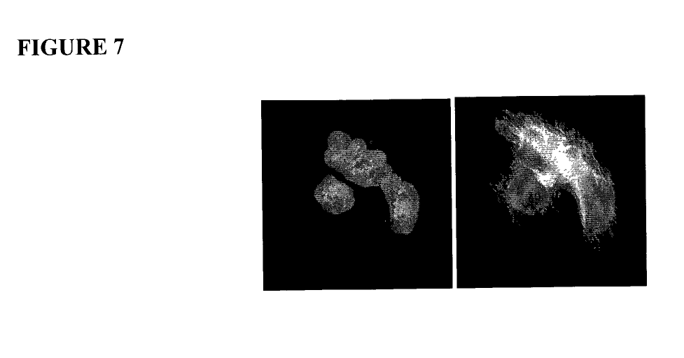

Corresponding phenotypes are also seen in Hela cells, where a 16-fold increase in multinucleate cells was observed (FIG. 7). The phenotype is consistent with a role in cytokinesis: in the absence of Ect2 nuclear division is completed, but the cleavage furrow and contractile ring do not form.

The above results confirm that Ect2 is involved in cell cycle progression, in particular, in achieving successful cell separation during cytokinesis. Accordingly, modulators of Ect2 activity (as identified by the assays described above) may be used to treat any proliferative disease.

### Example 6D

Expression of Ect2 in Tumour Cells

The level of expression of Ect2 in tumour and normal cells is determined using a cancer profile array (Clontech).

A specific Ect2 DNA probe is constructed by PCR amplification of a 612 bp region of an Ect2 cDNA clone (695-1306 bp). The DNA is purified following a standard PCR purification protocol (QIAquick PCR purification—Qiagen) before radioactive labelling using 6000 Ci/mmol α³²P [dCTP] (Amersham Pharmacia). This is done following a standard random hexamer labelling protocol (High prime—Roche).

Changes in expression level of Ect2 between normal and cancer cells are then detected by probing the cancer profile array with 100 ng of radiolabelled Ect2 probe. The radiolabelled DNA bound to the cancer profile array is detected by exposing the array to X-ray film for 6 days.

Analysis of the array data shows a change in the Ect2 expression level when compared to normal. Increases in expression level are seen for rectum (3/18 samples), lung (9/21 samples), ovary (10/14 samples), stomach (12/28 samples), colon (13/34 samples), uterus (10/42 samples), breast (6/50 samples), pancreas (1/1 samples) and small intestine tissue 1/2 samples).

A decrease in expression level was seen in rectum (3/18 samples), lung (1/21 samples) ovary (1/14 samples), colon (2/34) and uterus tissue (2/42). Expression of Ect2 expression was also found in nine cancer cell lines (Hela; Burkitt's lymphoma, Daudi; chronic myelogenous leukaemia K562; promyelocytic leukaemia HL-60; melanoma G361; lung carcinoma A549; lymphoblastic leukaemia MOLT-4; colorectal adenocarcinoma SW480 and Burkitt's lymphoma, Raji).

Thus modulators of Ect2 function may be particularly useful in treating diseases, such as proliferative diseases, or diseases involving misregulation of cellular proliferation, including those associated with any of the above tissues. In particular, modulators of Ect2 activity may be used to treat any of the following diseases: Burkitt's lymphoma, Daudi; chronic myelogenous leukaemia; promyelocytic leukaemia; melanoma; lung carcinoma; lymphoblastic leukaemia; colorectal adenocarcinoma and Burkitt's lymphoma, Raji.

### Example 6E

Expression of Recombinant Ect2 Protein in Insect Cells

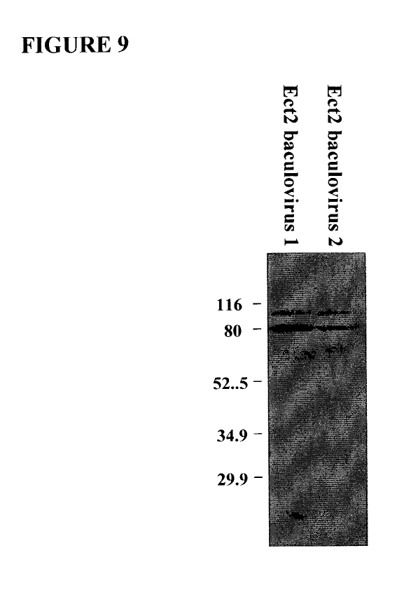

A cDNA encoding the Ect2 coding region is inserted into the BamHI site of baculovirus expression vector pFastbacHTc (Life Technologies). A baculovirus stock is generated and subsequent infections of Sf9 insect cells demonstrate expression of a N-terminal 6His tagged protein of approximately 90 kD, as shown in FIG. 9.

The recombinant protein is expressed and purified, and its expression detected by Western blot, as follows:

Expression of Recombinant Ect2 Protein in Insect Cells

A cDNA clone of Ect2 is created by RT-PCR of the full-length cDNA from Hela RNA, using a commercially available Long range RT-PCR kit (Life Technologies). The 2.7 kb PCR fragment is cloned into pCR4 TOPO (Invitrogen) and fully sequenced.

The full length Ect2 cDNA fragment is excised from the parent vector using a Bam HI restriction enzyme digestion and subcloned into the Bam HI site of the baculovirus expression vector pFastbac HTC (Life technologies). The Ect2 pFastbac vector is used to create a baculovirus stock following a standard protocol (Bac-TO-BAC Baculovirus Expression systems—Life technologies).

Briefly, the Ect2 pFastbac vector is transformed into DH10Bac competent cells containing a mini-attTn7 target site and a helper plasmid. The mini-Tn7 element of the pFastbac donor plasmid transposes to the mini-attTn7 target site on the bacmid and positive recombinant bacmid clones are identified by disruption of the LacZα gene. High molecular weight DNA is prepared from selected E. Coli clones following a standard protocol (Sambrook et. al; 1989) and used to infect Drosophila SF9 cells.

Bacmid DNA is transfected into 9×10⁵ SF9 cells using a mixture consisting of 94 μl Optiplex medium, 6 μl fugene 6 and 5 μl bacmid DNA. Cells are incubated for 3 days at 28° C. A small aliquot of infected cells is then used to infect a fresh culture of SF9 cells, and after a further 4 day incubation at 28° C. recombinant protein is purified from the cells following a standard protocol.

Recombinant Ect2 Western Blot

Baculovirus infected Drosophila SF9 cells are washed once in PBS before re-suspension in SDS-page loading buffer (100 mM Tris. Cl (pH 6.8); 200 mM DTT; 4% SDS; 20% Glycerol; 0.2% bromophenol blue). After boiling for 10 minutes, samples are loaded onto a 10% SDS-page gel and run for 90 minutes at 100V. Total protein is transferred to nitrocellulose (Schleicher and Schuell) following a standard protocol.

6-HIS tagged human Ect2 protein is detected using anti-HIS mouse antibody (1:2000) followed by rabbit anti-mouse peroxidase antibody (1:2000). Tagged protein is detected using a standard ECL protocol (Pierce).

Expression and Protein Purification

Recombinant protein is produced by cloning the Ect2 cDNA into a vector containing a HIS Tag (see above) and expressing the protein in E. Coli. Purification of the recombinant protein can be achieved by Ni-NTA purification.

### Example 6F

Assay for Modulators of Ect2 Activity

Modulators of Ect2 activity are identified by assaying activity of Ect2 in the presence of a candidate molecule. Ect2 activity is measured by utilising the ability of the protein to induce the exchange of GDP on the small GTPases RhoA, Rac1 and Cdc42 for GTP.

GST-RhoA or GST-Rac1 (Cytoskeleton Inc) is immobilised using an anti-GST coated microtitre plate. A sub-stoicheometric amount of [³H]-GDP is added and allowed to bind to the GST-RhoA or GST-Rac1. After equilibration Ect2 is added together with an excess of GTP, together with an amount of candidate modulator or test compound.

The exchange reaction is allowed to proceed at which point excess GTP and displaced [³H]-GDP are removed by washing. Scintillant is added and remaining [³H]-GDP is quantified by scintillation counting.

Effective modulators result in reduced exchange therefore retention of [³H]-GDP, and such molecules are identified and chosen for further study. A negative control is conducted as above but without the addition of Ect2.

Confirmation of Ect2 modulatory activity IS performed in an assay with the same format as described above but replacing Ect2 with another Rac1 or RhoA exchange factor (TIAM1 or VAV2).

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

References

Deak, P., Omar, M. M., Saunders, R. D. C., Pal, M., Komonyi, O., Szidonya, J., Maroy, P., Zhang, Y., Ashburner, M., Benos, P., Savakis, C., Siden-Kiamos, I., Louis, C., Bolshakov, V. N., Kafatos, F. C., Madueno, E., Modolell, J., Glover, D. M. (1997) Correlating physical and cytogenetic maps in chromosomal region 86E-87F of Drosophila melanogaster. Genetics 147:1697-1722.

Torok, T., Tick, G., Alvarado, M., Kiss, I. (1993) P-lacW insertional mutagenesis on the second chromosome of Drosophila melanogaster: isolation of lethals with different overgrowth phenotypes. Genetics 135(1):71-80

Saunders, R. D. C., Glover, D. M., Ashburner, M., Siden-Kiamos, I., Louis, C., Monastirioti, M., Savakis, C., Kafatos, F. C. (1989) PCR amplification of DNA microdissected from a single polytene chromosome band: a comparison with conventional microcloning. Nucleic Acids Res. 17:9027-9037

Lefevre, G. (1976) A photographic representation and interpretation of the polytene chromosomes of Drosophila melanogaster salivary glands. In: The Genetics and Biology of Drosophila, Eds Ashburner, M. and Novitski, E. Academic Press.

Jowett, T. (1986) Preparation of nucleic acids. In “Drosophila: A Practical Approach.” Ed Roberts, D. B. IRL Press Oxford.

Pirrotta, V. (1986) Cloning Drosophila genes. In: In “Drosophila: A Practical Approach.” Ed Roberts, D. B. IRL Press Oxford.

Altschul, S. F. and Lipman, D. J. (1990) Protein database searches for multiple alignments. Proc. Natl. Acad. Sci. USA 87: 5509-5513

Burge, C. and Karlin, S. (1997) Prediction of complete gene structures in human genomic DNA. J. Mol. Biol. 268, 78-94.

Each of the applications and patents mentioned above, and each document cited or referenced in each of the foregoing applications and patents, including during the prosecution of each of the foregoing applications and patents (“application cited documents”) and any manufacturer's instructions or catalogues for any products cited or mentioned in each of the foregoing applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A polynucleotide selected from:
(a) polynucleotides comprising any one of the nucleotide sequences set out in Examples 1 to 70 or the complement thereof.
(b) polynucleotides comprising a nucleotide sequence capable of hybridising to the nucleotide sequences set out in Examples 1 to 70, or a fragment thereof.
(c) polynucleotides comprising a nucleotide sequence capable of hybridising to the complement of the nucleotide sequences set out in Examples 1 to 70 or a fragment thereof.
(d) polynucleotides comprising a polynucleotide sequence which is degenerate as a result of the genetic code to the polynucleotides defined in (a), (b) or (c).

2. A polynucleotide selected from:
(a) polynucleotides comprising any one of the nucleotide sequences set out in Examples 1 to 14 or the complement thereof.
(b) polynucleotides comprising a nucleotide sequence capable of hybridising to the nucleotide sequences set out in Examples 1 to 14, or a fragment thereof.
(c) polynucleotides comprising a nucleotide sequence capable of hybridising to the complement of the nucleotide sequences set out in Examples 1 to 14 or a fragment thereof.
(d) polynucleotides comprising a polynucleotide sequence which is degenerate as a result of the genetic code to the polynucleotides defined in (a), (b) or (c).

3. A polynucleotide selected from:
(a) polynucleotides comprising any one of the nucleotide sequences set out in Examples 15 to 19 or the complement thereof.
(b) polynucleotides comprising a nucleotide sequence capable of hybridising to the nucleotide sequences set out in Examples 15 to 19, or a fragment thereof.
(c) polynucleotides comprising a nucleotide sequence capable of hybridising to the complement of the nucleotide sequences set out in Examples 15 to 19 or a fragment thereof.
(d) polynucleotides comprising a polynucleotide sequence which is degenerate as a result of the genetic code to the polynucleotides defined in (a), (b) or (c).

4. A polynucleotide selected from:
(a) polynucleotides comprising any one of the nucleotide sequences set out in Examples 20 to 30 or the complement thereof.
(b) polynucleotides comprising a nucleotide sequence capable of hybridising to the nucleotide sequences set out in Examples 20 to 30, or a fragment thereof.
(c) polynucleotides comprising a nucleotide sequence capable of hybridising to the complement of the nucleotide sequences set out in Examples 20 to 30 or a fragment thereof.
(d) polynucleotides comprising a polynucleotide sequence which is degenerate as a result of the genetic code to the polynucleotides defined in (a), (b) or (c).

5. A polynucleotide selected from:
(a) polynucleotides comprising any one of the nucleotide sequences set out in Examples 31 to 53 or the complement thereof.
(b) polynucleotides comprising a nucleotide sequence capable of hybridising to the nucleotide sequences set out in Examples 31 to 53, or a fragment thereof.
(c) polynucleotides comprising a nucleotide sequence capable of hybridising to the complement of the nucleotide sequences set out in 31 to 53 or a fragment thereof.
(d) polynucleotides comprising a polynucleotide sequence which is degenerate as a result of the genetic code to the polynucleotides defined in (a), (b) or (c).

6. A polynucleotide selected from:
(a) polynucleotides comprising any one of the nucleotide sequences set out in 54 to 70 or the complement thereof.
(b) polynucleotides comprising a nucleotide sequence capable of hybridising to the nucleotide sequences set out in 54 to 70, or a fragment thereof.
(c) polynucleotides comprising a nucleotide sequence capable of hybridising to the complement of the nucleotide sequences set out in 54 to 70 or a fragment thereof.
(d) polynucleotides comprising a polynucleotide sequence which is degenerate as a result of the genetic code to the polynucleotides defined in (a), (b) or (c).

7. A polynucleotide probe which comprises a fragment of at least 15 nucleotides of a polynucleotide according to any of claims 1 to 6.

8. A polypeptide which comprises any one of the amino acid sequences set out in Examples 1 to 70 or in any of Examples 1 to 14, Examples 15 to 19, Examples 20 to 30, Examples 31 to 53 and Examples 54 to 70, or a homologue, variant, derivative or fragment thereof.

9. A polynucleotide encoding a polypeptide according to claim 8.

10. A vector comprising a polynucleotide according to any of claims 1 to 7 and 9.

11. An expression vector comprising a polynucleotide according to any of claims 1 to 7 and 9 operably linked to a regulatory sequence capable of directing expression of said polynucleotide in a host cell.

12. An antibody capable of binding a polypeptide according to claim 8.

13. A method for detecting the presence or absence of a polynucleotide according to any of claims 1 to 7 and 9 in a biological sample which comprises:
(a) bringing the biological sample containing DNA or RNA into contact with a probe according to claim 9 under hybridising conditions; and
(b) detecting any duplex formed between the probe and nucleic acid in the sample.

14. A method for detecting a polypeptide according to claim 8 present in a biological sample which comprises:
(a) providing an antibody according to claim 12;
(b) incubating a biological sample with said antibody under conditions which allow for the formation of an antibody-antigen complex; and
(c) determining whether antibody-antigen complex comprising said antibody is formed.

15. A polynucleotide according to according to any of claims 1 to 7 and 9 for use in therapy.

16. A polypeptide according to claim 8 for use in therapy.

17. An antibody according to claim 12 for use in therapy.

18. A method of treating a tumour or a patient suffering from a proliferative disease comprising administering to a patient in need of treatment an effective amount of a polynucleotide according to any of claims 1 to 7 and 9.

19. A method of treating a tumour or a patient suffering from a proliferative disease, comprising administering to a patient in need of treatment an effective amount of a polypeptide according to claim 8.

20. A method of treating a tumour or a patient suffering from a proliferative disease, comprising administering to a patient in need of treatment an effective amount of an antibody according to claim 12 to a patient.

21. Use of a polypeptide according to claim 8 in a method of identifying a substance capable of affecting the function of the corresponding gene.

22. Use of a polypeptide according to claim 8 in an assay for identifying a substance capable of inhibiting the cell division cycle.

23. Use as claimed in claim 22, in which the substance is capable of inhibiting mitosis and/or meiosis.

24. A method for identifying a substance capable of binding to a polypeptide according to claim 8, which method comprises incubating the polypeptide with a candidate substance under suitable conditions and determining whether the substance binds to the polypeptide.

25. A method for identifying a substance capable of modulating the function of a polypeptide according to claim 8 or a polypeptide encoded by a polynucleotide according to any of claims 1 to 7 and 9, the method comprising the steps of: incubating the polypeptide with a candidate substance and determining whether activity of the polypeptide is thereby modulated.

26. A substance identified by a method or assay according to any of claims 21 to 25.

27. Use of a substance according to claim 26 in a method of inhibiting the function of a polypeptide.

28. Use of a substance according to claim 26 in a method of regulating a cell division cycle function.
